(19) United States
(12) Patent Application Publication
(10) Pub. No.: US 2025/0009866 A1
(43) Pub. Date: Jan. 9, 2025

(54) MODULATION AND UTILIZATION OF ENANTIOMER-DEPENDENT IMMUNOLOGICAL RESPONSE TO CHIRAL NANOPARTICLES

(71) Applicants: The Regents of the University of Michigan, Ann Arbor, MI (US); JIANGNAN UNIVERSITY, Wuxi, Jiangsu (CN)

(72) Inventors: Nicholas KOTOV, Ypsilanti, MI (US); Hua KUANG, Wuxi, Jiangsu (CN); Liguang XU, Wuxi, Jiangsu (CN); Chuanlai XU, Wuxi, Jiangsu (CN)

(21) Appl. No.: 18/712,516

(22) PCT Filed: Nov. 22, 2022
(86) PCT No.: PCT/US22/50768

§ 371 Date: May 22, 2024

Related U.S. Application Data
(60) Provisional application No. 63/282,100, filed on Nov. 22, 2021.

(51) Int. Cl. A61K 39/12; A61K 9/14; A61K 39/00; A61K 39/02; A61K 39/39; A61P 31/16

(52) U.S. Cl. CPC A61K 39/12; A61K 9/14; A61K 39/0011; A61K 39/02; A61K 39/39; A61P 31/16; A61K 2039/545; A61K 2039/55505

(57) ABSTRACT

A method for modulation of an immune response utilizing chiral nanoparticles. The chiral nanoparticles can be synthesized from nanoparticle seed precursors using circularly polarized light. The chirality of the NPs can be measured using chirality measures such as the Osipov-Pickup-Dunmeur index, the Hausdorff chirality measure and by using circular dichroism spectra, g-factor spectra, and maximum g-factor for a specific wavelength. In-vivo tests in mice showed a 2.27-fold enhancement of immune cell maturation and 1584-fold enhancement of IgG production by L-nanoparticles versus D-nanoparticles. Both the in-vivo and in-vitro immune responses monotonically depend on the chirality, measured as g-factors, of the nanoparticles, indicating that nanoscale chirality of the nanoparticles can be used to regulate immune cell maturation. Finally, L-nanoparticles demonstrated 1258-fold higher efficiency as adjuvants for the H9N2 influenza virus versus D-nanoparticles or Alum, opening a path to utilization of nanoscale chirality in immunology.

### STATEMENT REGARDING FEDERALLY SPONSORED RESEARCH

This invention was made with government support under CMMI1463474 awarded by the National Science Foundation. The government has certain rights in the invention.

### TECHNICAL FIELD

This present disclosure relates generally to chiral nanoparticles, and more particularly to their use in immunological responses.

### BACKGROUND

This section provides background information which is not necessarily prior art to the inventive concepts associated with the present disclosure.

Chirality is a unifying structural metric of biological and abiological forms of matter. In the last decade, considerable clarity was achieved in the understanding of the chemistry and physics of chiral inorganic nanoparticles (NPs), however, little is still known about their effects on complex biochemical networks. Lock-and-key interactions between mirror-symmetrical molecules are known to encode complex sequences of cell signaling events, including immune system responses. Left- and right-handed chiral amino acids, proteins, and oligodeoxynucleotides can cause markedly different immune responses, but whether the same is true for NPs with opposite handedness is unknown. The present inventors are unaware of any use of chiral nanoparticles in any vaccines or other immunological treatments of infectious, oncological or autoimmune diseases. While intermolecular interactions of biomolecules and NPs display some commonalities, they differ in scale, geometry, and dynamics of chiral shapes, which can both impede and strengthen the mirror-asymmetric complexes.

Chiral inorganic nanostructures, obtained by a variety of methods, have been fueling discoveries in optoelectronics, sensing, and enantioselective catalysis because of their strong chiroptical activity and ability to self-assemble. However, little is known about the effects of nanoscale chirality on complex system responses of live organisms. Randomly shaped, nearly spherical, and achiral inorganic NPs activate the immune system. But chiral NPs may allow for a particularly tight ‘lock-and-key configuration with cell receptors. See Nicholas A. Kotov, Inorganic Nanoparticles as Protein Mimics, Science, Vol. 330, Issue 6001 (8 Oct. 2010). Furthermore, the chirality of the particles attained in different chemical pathways may modulate their immunological properties because protein-protein complexes governing the immune responses also have nanoscale dimensions and mirror asymmetry. At the same time, recognition of NP enantiomers by the immune system may be drastically impeded by the rigidity of the inorganic NP cores because dynamic adaptation of complex shapes of the biomolecules is often required for lock-and-key interactions. Formation of protein coronas may also ‘camouflage’ the asymmetry of particle core geometry. The study of immune cell activation by NPs with strong mirror asymmetry would shed light on the role of nanoscale chirality in system-level biological responses and methods for chirality-based design of nanoscale vaccine adjuvants.

### SUMMARY

This section provides a general summary of the present disclosure and is not intended to be interpreted as a comprehensive disclosure of its full scope or all features, aspects and objectives.

The present disclosure shows that chiral, left- and right-handed inorganic NPs exemplified but not limited to gold-based chiral NPs, display different in-vitro and in-vivo immune responses. In one specific implementation not limiting the scope of chiral nanostructures, chirality of the NPs was imparted by irradiation with circularly polarized light (CPL), which affords NPs with controllable nanoscale chirality and optical g-factors up to 0.4. Preferably, the chiral NPs according to the present invention have absolute g-factor values of 0.00001 or greater, 0.0001 or greater, 0.001 or greater, 0.01 or greater, more preferably 0.1 or greater, more preferably 0.2 or greater, more preferably 0.3 or greater, most preferably 0.4 or greater. As known to one of skill in the art the notation +/−before a g-factor value indicates that it refers to left or right polarization rotation specifically. If no sign is used in front of the g-factor value, then this value refers to an absolute value of this parameter meaning a g-factor value independent of whether it is left or right polarization. Thus, a g-factor value of −0.0001 is greater than a g-factor value of −0.00001. Preferably, the chiral NPs have an absolute Osipov-Pickup-Dunmeur chirality index (OPD) value of greater than 0, or a Hausdorff chirality measure (HCM) value of greater than 0.

Optical anisotropy factor, also known as g-factor, can quantitatively characterize the optical activity of chiral NPs. Both mouse bone marrow dendritic cells (BMDCs) and mouse bone-marrow-derived macrophages (BMMs) displayed higher uptake of left-handed NPs than right-handed NPs because of higher binding affinity to two proteins from the adhesion G protein-coupled receptor family (AGPCR), namely, the cluster of differentiation 97 (CD97) marker and epidermal growth factor-like module receptor 1 (EMR1). Binding of NPs to these two proteins results in the opening of mechanosensitive K⁺ efflux channels, subsequent production of inflammasomes, and maturation of mouse BMDCs. The inflammasomes are innate immune system cytosolic multiprotein oligomers that act as receptors and sensors to regulate the activation of caspase-1 and induce inflammation in response to infectious microbes and molecules derived from host proteins. Higher affinity of binding to these proteins by left-handed NPs generates substantially enhanced downstream in-vitro immune response from the left-handed NPs versus from right-handed NPs. In-vivo tests in mice showed a 2.27-fold enhancement of immune cell maturation and 1584-fold enhancement of IgG production for left-handed NPs versus for right-handed NPs. Both the in-vivo and in-vitro immune responses monotonically depend on the g-factors of the NPs, indicating that nanoscale chirality of the NPs can be used to regulate immune cell maturation. Finally, left-handed NPs demonstrated significantly higher, 1258-fold, efficiency as adjuvants for the H9N2 influenza virus versus the right-handed NPs, opening a path to utilization of nanoscale chirality in immunology.

In one aspect the present disclosure provides a method of producing chiral gold nanoparticles comprising the steps of. a.) providing a plurality of gold nanoprism seeds having an average edge length of 70 to 80 nm; b.) preparing a growth solution comprising cetyltrimethylammonium bromide (CTAB), hydrogen tetrchloroaurate (HAuCl4) and ascorbic acid in distilled water; c.) adding the nanoprism seeds and a plurality of one dipeptide selected from the group consisting of L-cysteine-phenylalanine and D-cysteine-phenylalanine to the growth solution to form a reaction solution; and d.) illuminating the reaction solution with right circularly polarized light when selecting as the dipeptide D-cysteine-phenylalanine or left circularly polarized light when selecting as the dipeptide L-cysteine-phenylalanine at a wavelength of 594 nm and an intensity of 84 mW/cm² for a period of time, thereby forming a plurality of chiral nanoparticles having a handedness that is the same as that of the selected dipeptide.

In another aspect the present disclosure provides a vaccine adjuvant comprising a chiral inorganic nanoparticle having a g-factor value of +/−0.00001 or greater, +/−0.0001 or greater, +/−0.001 or greater, +/−0.01 or greater, more preferably +/−0.1 or greater, more preferably +/−0.2 or greater, more preferably +/−0.3 or greater, most preferably +/−0.4 or greater. Preferably, the chiral NPs have an absolute Osipov-Pickup-Dunmeur chirality index (OPD) value of greater than 0, or a Hausdorff chirality measure (HCM) value of greater than 0.

In another aspect the present disclosure provides a method of inducing an immune response to an antigen in a subject comprising administering to the subject an adjuvant comprising a chiral gold nanoparticle having a g-factor value of +/−0.00001 or greater, +/−0.0001 or greater, +/−0.001 or greater, +/−0.01 or greater, more preferably +/−0.1 or greater, more preferably +/−0.2 or greater, more preferably +/−0.3 or greater, most preferably +/−0.4 or greater. Preferably, the chiral NPs have an absolute Osipov-Pickup-Dunmeur chirality index (OPD) value of greater than 0, or a Hausdorff chirality measure (HCM) value of greater than 0.

In another aspect the present disclosure provides a vaccine composition comprising: (a) an antigen selected from the group consisting of influenza, Covid 19, polio, rubella, mumps, chicken, ebola, hepatitis B, human papilloma, tuberculosis, diphtheria, pertussis (whooping cough), tetanus, rotavirus, hepatitis A, Haemophilus influenzae type b (Hib), rabies, RTS,S/ASO1 (Mosquirix™), shingrix, hepatitis C, HIV, SIV, dengue virus, West Nile, zika virus, herpes simplex virus, human cytomegalovirus, respiratory syncytial virus, adenovirus, vesicular stomatitis virus, encephalomyocarditis virus, Norovirus, anthrax, measles, typhoid, cholera, and diphtheria; and (b) an adjuvant comprising a chiral nanoparticle having a g-factor of +/−0.00001 or greater, +/−0.0001 or greater, +/−0.001 or greater, +/−0.01 or greater, more preferably +/−0.1 or greater, more preferably +/−0.2 or greater, more preferably +/−0.3 or greater, most preferably +/−0.4 or greater. Preferably, the chiral NPs have an absolute Osipov-Pickup-Dunmeur chirality index (OPD) value of greater than 0, or a Hausdorff chirality measure (HCM) value of greater than 0.

In another aspect the present disclosure provides a method of inducing an immune response in a subject comprising administering a vaccine to the subject in need thereof, said vaccine comprising: (a) an antigen selected from the group consisting of influenza, Covid 19, polio, rubella, mumps, chicken, ebola, hepatitis B, human papilloma, tuberculosis, diphtheria, pertussis (whooping cough), tetanus, rotavirus, hepatitis A, Haemophilus influenzae type b (Hib), rabies, RTS,S/ASO1 (Mosquirix™), shingrix, hepatitis C, HIV, SIV, dengue virus, West Nile, zika virus, herpes simplex virus, human cytomegalovirus, respiratory syncytial virus, adenovirus, vesicular stomatitis virus, encephalomyocarditis virus, Norovirus, anthrax, measles, typhoid, cholera, and diphtheria; and (b) an adjuvant comprising a chiral gold nanoparticle having a g-factor of +/−0.00001 or greater, +/−0.0001 or greater, +/−0.001 or greater, +/−0.01 or greater, more preferably +/−0.1 or greater, more preferably +/−0.2 or greater, more preferably +/−0.3 or greater, most preferably +/−0.4 or greater. Preferably, the chiral NPs have an absolute Osipov-Pickup-Dunmeur chirality index (OPD) value of greater than 0, or a Hausdorff chirality measure (HCM) value of greater than 0.

These and other features and advantages of this disclosure will become more apparent to those skilled in the art from the detailed description herein. The drawings that accompany the detailed description are described below.

### BRIEF DESCRIPTION OF THE DRAWINGS

The drawings described herein are for illustrative purposes only of selected aspects and not all implementations, and are not intended to limit the present disclosure to only that actually shown. With this in mind, various features and advantages of example aspects of the present disclosure will become apparent to one possessing ordinary skill in the art from the following written description and appended claims when considered in combination with the appended drawings, in which:

FIG. 1A is a scanning electron microscope photograph of gold nanoparticles prepared using L-cysteine-phenylalanine without any illumination; FIG. 1B is a scanning electron microscope photograph of gold nanoparticles prepared using D-cysteine-phenylalanine without any illumination; FIG. 1C is a 3D reconstruction transmission electron microscopy tomography image of a gold nanoparticle prepared using L-cysteine-phenylalanine without any illumination; and FIG. 1D is a 3D reconstruction transmission electron microscopy tomography image of a gold nanoparticle prepared using D-cysteine-phenylalanine without any illumination;

FIG. 2 shows the effect of illumination wavelength for linearly polarized light and intensity of the light on chirality as shown by the measured g-factor of formed L-P⁰NPs prepared from gold nanoprism seeds according to the present disclosure;

FIG. 3 is a series of Scanning Electron Microscope (SEM) images of the growth of L-P⁺NPs after 0, 5, 10, 20, 30 and 40 minutes of left circularly polarized illumination at 594 nm at an intensity of 84 mW/cm² according to the present disclosure;

FIGS. 4A and 4B show the circular dichroism spectra and g-factor spectra, respectively, of L-P⁺NPs after 0, 5, 10, 20, 30 and 40 minutes of left circularly polarized illumination at 594 nm at an intensity of 84 mW/cm² according to the present disclosure;

FIG. 5A is an SEM image of nanoscale enantiomers of L-P⁺NPs formed after 30 minutes of left circularly illumination at 594 nm at an intensity of 84 mW/cm2 according to the present disclosure while FIG. 5B is an SEM image of nanoscale enantiomers of D-P⁺NPs formed after 30 minutes of right circularly polarized illumination at 594 nm at an intensity of 84 mW/cm2 according to the present disclosure;

FIG. 6 shows Transmission Electron Microscope (TEM) tomography images of the following nanoparticles prepared according to the present disclosure L-P⁺NP, D-P⁻NP, L-P⁻NP and L-P⁰NP from nanoprism seeds using polarized illumination of the indicted direction at 594 nm, an intensity of 84 mW/cm² and with the indicated surface ligands;

FIG. 7A shows five, ((i) to (v)), calculated stages of gold deposition based on dynamic formation of “hot spots” on the surface of D-P⁻NPs grown from nanoprism seeds under illumination by right circularly polarized light; FIG. 7B shows the electric field distribution for stage (i) of FIG. 7A; FIG. 7C shows the electric field distribution for stage (iii) of FIG. 7A; and FIG. 7D shows the electric field distribution for stage (iv) of FIG. 7A;

FIG. 8A is an SEM photograph of L-NP-C nanoparticles that were grown from nanocubes as the seeds without illumination, using L-cysteine-phenylalanine as the surface ligand; FIG. 8B is an SEM photograph of L-P⁺NP-C nanoparticles that were grown from nanocubes with illumination at 532 nm an intensity of 84 mW/cm² using L-CYP as the surface ligand for 30 minutes; FIG. 8C shows the circular dichroism spectra for the nanoparticles L-NP-C, L-P⁺NP-C, D-NP-C and D-P⁻NP-C; and FIG. 8D shows the g-factor spectra for L-NP-C, L-P⁺NP-C, D-NP-C and D-P⁻NP-C derived from FIG. 8C;

FIG. 9A is an SEM photograph of L-NP-O nanoparticles that were grown from octahedrons as the seeds without illumination, using L-cysteine-phenylalanine as the surface ligand; FIG. 9B is an SEM photograph of L-P⁺NP-O nanoparticles that were grown from octahedrons with illumination at 532 nm an intensity of 84 mW/cm² using L-CYP as the surface ligand for 30 minutes; FIG. 9C shows the circular dichroism spectra for L-NP-O, L-P⁺NP-O, D-NP-O and D-P⁻NP-O; and FIG. 9D shows the g-factor for L-NP-O, L-P⁺NP-O, D-NP-O and D-P⁻NP-O derived from FIG. 9C;

FIG. 10 shows the simulated circular dichroism by FDTD, solids lines, and the a circular dichroism, dot-dash lines, from the NP model shown in FIG. 7A at stage (v) using Limerical software and differences in extinction coefficients of L-P⁺NPs and D-P⁻NPs models obtained by electrodynamics calculations;

FIGS. 11A to 11C show the modeling used to calculate the chirality measures of L-P⁺NPs based on the TEM tomography images of L-P⁺NPs from FIG. 6, dividing the L-P⁺NPs with the octants of the coordinate system is shown in FIG. 11B-11C;

FIGS. 12A to 12C show the modeling used to calculate the chirality measures of D-P⁻NPs based on the TEM tomography images of D-P⁻NPs from FIG. 6, dividing the D-P⁺NPs with the octants of the coordinate system is shown in FIG. 12B-12C;

FIG. 13 shows two-photon luminescence staining for 4′,6-diamidino-2-phenylindole (DAPI) and chiral nanoparticles of cultured mouse bone marrow dendritic cells incubated with either L-P⁺NPs or D-P⁻NPs at 2 nM for the indicated time periods, FIG. 13A are for L-P⁺NPs and FIG. 13B are for L-P⁺NPs, scale bar is 10 micrometers;

FIG. 14 shows the uptake of L-P⁺NPs (solid squares) or D-P⁻NPs (solid circles) over time as measured by absorbance (solid lines) or circular dichroism (dotted lines) in cultured mouse bone marrow dendritic cells incubated with either L-P⁺NPs or D-P⁻NPs at 2 nM for the indicated time periods;

FIGS. 15A and 15B show two-photon luminescence staining for 4′,6-diamidino-2-phenylindole (DAPI) and chiral nanoparticles of cultured mouse bone marrow dendritic cells incubated with either L-P⁺NPs (A) or D-P⁻NPs (B) at 2 nM for the indicated time periods, scale bars are 20 micrometers, FIG. 15 C shows quantification of the gray value of the white dots from the L-P⁺NPs (A) or D-P⁻NPs at each time point with the first bar of each time point in 15C being the L-P⁺NPs and the second bar being the D-P⁻NPs, the *** is significance at P<0.001 and **** is significance at P<0.0001 calculated by one-way ANOVA;

FIG. 16 shows the results of exposing mouse BMDCs to L-type NPs (solid squares) or D-type NPs (open squares) with varied g-factor values for 12 hours at 2 nM and then CD86 plus cells were measured by flow cytometry, the data shown are mean±s.d., n=5, and * P<0.005, ** P<0.01, *** P<0.001 analyzed by Student's t-test, the g-factor 0.0 data point are the nanoprisms;

FIGS. 17A, 17B, and 17C show in vivo expression of CD40, CD80, and CD86, respectively, in recruited dendritic cells, identified by the CD11c⁺ marker, from mouse draining lymph node cells 36 hours after injection of the mice with 2 mg of the chiral LID NPs having a variety of g-factor values, 10 μg of MPL and 50 μg of OVA, analyzed by flow cytometry, the solid symbols are the L-P^{+X}NPs and the open symbols are the D-P^{-X}NPs, with g-factor 0.0 being nanoprism seeds;

FIG. 18A shows measured secretion of interferon-gamma (IFN-γ) by CD4⁺ spleen T cells isolated from mice 7 days after immunization with the indicated substances, and FIG. 18B shows measured secretion of interferon-gamma (IFN-γ) by CD8+spleen T cells isolated from mice 7 days after immunization with the indicated substances;

FIG. 19 shows the OVA-specific IgG titer levels in mouse serum over 91 days with immunization with OVA plus MPL and the indicated adjuvant at time 0, day 14 and day 56, note the Y-axis is a logarithmic scale Log₁₀, the black arrows indicate the times of immunization;

FIG. 20A shows a schematic of a possible interaction between a chiral nanoparticle according to the present disclosure and an extracellular segment of up to 5-6 epidermal growth factor (EGF) like domains found on epidermal growth factor-like module receptor 1 (EMR1) and the receptor differentiation 97 (CD97 while FIG. 20B shows a schematic of the possible in vivo mechanism of induction of immune responses by chiral nanoparticles according to the present disclosure;

FIGS. 21A to 21D show Isothermal Titration Calorimetry (ITC) data for L-P⁺NP (100 μM) and for D-P⁻NP (100 μM) after incubation with CD97 (10 μM) and EMR1 (10 μM) for 30 minutes, FIG. 21A shows data for L-P⁺NP binding to CD97, FIG. 21B shows data for D-P⁻NP binding to CD97, FIG. 21C shows data for L-P⁺NP binding to EMR1, and FIG. 21D shows data for D-P⁻NP binding to EMR1;

FIGS. 22A and 22B show the calculated binding affinity (Ka) data for a series of L-P^{+X}NPs and D-P^{-X}NPs having different g-factor values binding to CD97 and EMR1, respectively, note the Y-axis for binding to CD97 is split to allow for accommodation of the high binding affinity of L-P^{+X}NPs to fit on the graph, solid symbols are L-P^{+X}NPs and open symbols are D-P^{-X}NPs;

FIG. 23A and FIG. 23B show uptake of L-P⁺NPs and D-P⁻NPs, respectively, by mouse BMDCs monitored by flow cytometry, the results are presented as relative mean fluorescence intensity (MFI) %, the control uptake was in PBS and the other data show the effects on uptake of blocking CD97 receptors, blocking EMR1 receptors, blocking both CD97 and EMR1 receptors, blocking phagocytosis, blocking microtubules, blocking clathrin or blocking dynamin;

FIG. 24 shows uptake of L-P⁺NPs and D-P⁻NPs, respectively, by human BMDCs monitored by flow cytometry, the results are presented as relative mean fluorescence intensity (MFI) %, the control uptake was in PBS and the other data show the effects on uptake of blocking CD97 receptors, blocking EMR1 receptors, or blocking both CD97 and EMR1 receptors;

FIG. 25, panels A-F show confocal imaging of NLRP3 inflammasome activation in mouse BMDCs after incubated with PBS(25A), MPL+OVA (25B), L-P⁺NP+MPL+OVA (25C), L-P⁺NP+MPL+OVA+MCC950 (NLRP3 inhibitor) (25D), L-P⁺NP+MPL+OVA+amiodarone (K⁺ channel inhibitor) (25E), and L-P⁺NP+MPL+OVA+KCl (K⁺ efflux inhibitor) (25F) for 12 hours;

FIG. 26, panels A-F show confocal imaging of NLRP3 inflammasome activation in mouse BMDCs after incubated with, L-P⁺NP+MPL+OVA+dynasore (dynamin inhibitor) (26A), L-P⁺NP+MPL+OVA+chlorpromazine (clathrin inhibitor) (26B), L-P⁺NP+MPL+OVA+CA-074-Me (cathepsin B inhibitor) (26C), L-P⁺NP+MPL+OVA+cytochalasin D (phagocytosis inhibitor) (26D), L-P⁺NP+MPL+OVA+NAC (reactive oxygen species (ROS) inhibitor) (26E) and L-P⁺NP+MPL+OVA+nocodazole (microtubule inhibitor) (26F) for 12 hours;

FIG. 27 shows Western blot data for the expression of NLRP3, IL-10, and caspase-1 in BMDCs after exposure to the indicated L-P^{+X}NPs or D-P^{-X}NPs at 2 nM plus 2 μg/mL MPL and 20 μg/mL OVA;

FIG. 28 shows flow cytometry results of the effect of subcutaneous injection of 2 mg of the indicated NPs, MPL (10 μg), and OVA (50 μg) on expression of NLRP3 in mouse draining lymph nodes 36 hours after injection;

FIG. 29 shows the H9N2 specific serum IgG titers over a 91 day period for C57BL/6 mice immunized with H9N2 influenza vaccine (10⁸ ELD₅₀/0.1 mL, 60 μL) plus the indicated adjuvants: 2 mg L-P⁺NP+10 μg MPL, 2 mg of D-P⁻NP+10 μg MPL, 50 μL Alum (Thermo Fisher Scientific, 77161)+10 μg MPL, or PBS+10 μg MPL, for three times, day 0, day 14, and day 56, the titers were measured by ELISA; and

FIGS. 30A to 30C show responses by flow cytometry measurement for IFN-γ-secreting CD8⁺ T cells, IFN-γ-secreting CD4⁺ T cells and IL-4-secreting CD4⁺ T cells isolated from the spleens of C57BL/6 mice 7 days after they were immunized with H9N2 influenza vaccine with the indicated adjuvants, including MPL, Alum+MPL, D-P⁻NP+MPL, L-P⁺NP+MPL, NS-D-CYP+MPL, or NS-L-CYP+MPL.

### DETAILED DESCRIPTION

In the following description, details are set forth to provide an understanding of the present disclosure.

For clarity purposes, example aspects are discussed herein to convey the scope of the disclosure to those skilled in the relevant art. Numerous specific details are set forth such as examples of specific components, devices, and methods, in order to provide a thorough understanding of various aspects of the present disclosure. It will be apparent to those skilled in the art that specific details need not be discussed herein, such as well-known processes, well-known device structures, and well-known technologies, as they are already well understood by those skilled in the art, and that example embodiments may be embodied in many different forms and that neither should be construed to limit the scope of the disclosure.

The terminology used herein is for the purpose of describing particular example aspects only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or feature is referred to as being “on,” “engaged to,” “connected to,” “coupled to” “operably connected to” or “in operable communication with” another element or feature, it may be directly on, engaged, connected or coupled to the other element or layer, or intervening elements or features may be present. In contrast, when an element is referred to as being “directly on,” “directly engaged to,” “directly connected to,” or “directly coupled to” another element or feature, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly and expressly indicated by the context. Thus, a first element, component, region, layer or section discussed below could be termed a second element, component, region, layer or section without departing from the teachings of the example embodiments.

For purposes of description herein, the terms “upper,” “lower,” “right,” “left,” “rear,” “front,” “vertical,” “horizontal,” and derivatives thereof shall relate to the invention as oriented in the FIGS. However, it is to be understood that the present disclosure may assume various alternative orientations and step sequences, except where expressly specified to the contrary. It is also to be understood that the specific devices and processes illustrated in the attached drawings, and described in the following specification are exemplary aspects of the inventive concepts defined in the appended claims. Hence, specific dimensions and other physical characteristics relating to the aspects disclosed herein are not to be considered as limiting, unless the claims expressly state otherwise.

The following is a list of some of the abbreviations used throughout the specification and claims: inorganic nanoparticles (NPs); circularly polarized light (CPL); left circularly polarized light (LCPL); right circularly polarized light (RCPL); linearly polarized light (LPL); mouse bone marrow dendritic cells (BMDCs); mouse bone-marrow-derived macrophages (BMMs); the adhesion G protein-coupled receptor family (AGPCR); the protein cluster of differentiation 97 (CD97) marker; epidermal growth factor-like module receptor 1 (EMR1); scanning electron microscopy (SEM); transmission electron microscopy (TEM); energy-dispersive X-ray spectroscopy (EXD); selected area electron diffraction (SAED); molar concentration (M); millimolar concentration (mM); nanomolar concentration (nM); milliliter (mL); microliter (L); nanometer (nm); nanogram (ng), milliwatt (mW); monophosphoryl lipid A (MPL); ovalbumin (OVA); the dipeptide cysteine-phenylalanine (CYP), either L or D form; Interleukin-12 (IL-12); Interleukin-1β (IL-1β); the Osipov-Pickup-Dunmeur chirality index (OPD). Below Table 1 provides a list of abbreviations to nanoparticles employed in this disclosure.

Nanoparticles display both molecular chirality and nanoscale chirality, corresponding to the geometry of any surface ligands and the geometry of the nanoparticles as a whole, respectively. Both scales of chirality can play a role in cell signaling network activation. Because a typical synthesis of chiral NPs involves coupled molecular and nanoscale chirality, it is, however, difficult to unambiguously assign the biological effects to one or the other. To address this problem, in this disclosure the inventors utilized a photosynthetic method for preparing enantiopure gold NPs via illumination of gold seed particles with circularly polarized light (CPL) and linearly polarized light (LPL). The degree of NP asymmetry can be varied by changing the parameters of illumination while keeping the chemical parameters constant. Gold nanoprisms and other achiral shaped gold nanoparticle seeds, stabilized by chiral surface ligands, were used as seeds to form the final chiral left or right-handed NPs. The NPs synthesized under CPL illumination in the presence of different dipeptides are herein referred to as L/D-P^{X}NPs wherein: LID stands for the chirality of the L/D-dipeptide surface ligand used to stabilize the seeds; X≡(+, −, 0) denotes the polarization of the photons used in the synthesis of the nanoparticles from the seeds, in which + represents illumination conditions with left circularly polarized light (LCPL), − represents illumination with right circularly polarized light (RCPL), and 0 represents the synthesis under linearly polarized light (LPL). The synthesis of chiral gold particles with controlled chirality in the present disclosure is based on a photon-to-particle chirality transfer mechanism.

### EXPERIMENTAL METHODS

### Reagents

The reagents used in the studies described in this disclosure were obtained from the following sources. Hydrogen tetrachloroaurate trihydrate (HAuCl₄·3H₂O), cetyltrimethylammonium bromide (CTAB, purity ≥99%), ascorbic acid (purity ≥99.9%), and ethylene glycol (EG, purity ≥99%) were obtained from Sigma-Aldrich. Poly (diallyldimethylammonium chloride) (PDDA) solution (MW=400,000-500,000, 20 wt % in H20) and L-Cys-methyl ester hydrochloride were obtained from Aladdin. Penicillamine (Pen, purity ≥98%) and hexadecyl trimethyl ammonium chloride (CTAC, purity ≥95.0%) were obtained from TCI Chemical Industries. Potassium iodide (KI, purity ≥99%), sodium borohydride (NaBH₄, purity ≥96%), hydrochloric acid (HCl), and sodium hydroxide (NaOH, purity ≥95%) were purchased from Sinopharm Chemical Reagent Co., Ltd. All amino acids used in this research were purchased from Sigma-Aldrich, unless otherwise stated. The dipeptides penicillamine-phenylalanine (purity ≥95%), cysteine-tyrosine (purity ≥97%), cysteine-tryptophan (purity ≥75%), and cysteine-histidine (purity ≥70%) were purchased from ChinaPeptides, Co., Ltd. Other dipeptides (purity ≥98%), were purchased from Sangon Biotechnology. All aqueous solutions were prepared with deionized water (18.2 MO; Millipore). The 532 nm, 594 nm, 660 nm, and 808 nm lasers and quarter wave plate for 532 nm, 660 nm, and 808 nm were purchased from Changchun New Industries Optoelectronics Technology Co., Ltd. Quarter wave plate for 594 nm was purchased from Thorlabs. The methoxy poly(ethylene glycol) thiol (mPEG-SH) had a molecular weight of 2000 and was obtained from Sinopharm Chemical Reagent Co, Ltd. (Shanghai, P.R. China).

### Detection/Analysis Methods

Detection and analysis methods and instrumentation used in the following disclosure were as follows. Circular dichroism spectra were obtained using a Chirascan-Plus circular dichroism spectrometer, and the spectra were processed using the OriginLab® software. The measurement temperature was maintained at 25° C. The scanning range for all samples was 300-1000 nm under high-purity nitrogen. Transmission electron microscopy (TEM) images were obtained with a transmission electron microscope, JEOL JEM-2100, operating at an acceleration voltage of 200 kV. The 3D reconstruction TEM tomography images and selected area electron diffraction (SAED) images were obtained with a 200 kV field emission electron microscope, FEI Tecnai F20. The energy-dispersive X-ray spectroscopy (EDX) mapping images and atomic structure images were characterized using a JEM ARM-200, JEOL, Tokyo, Japan, transmission electron microscope operated at 200 kV. The atomic-resolved high-angle annular dark-field (HAADF) images were acquired with a collection angle of 90-370 mrad. Scanning electron microscopy (SEM) images were obtained with a HITACHI Regulus 8220 and Hitachi S-4700. Dynamic light scattering (DLS) and zeta potential results were obtained by using a Zetasizer Nano ZS system, Malvern Instruments Ltd., Malvern, UK, with a 633 nm laser. Reverse transcription polymerase chain reaction (RT-PCR) experiments were performed on the Applied Biosystems 7900HT Fast Real-Time PCR System, Waltham, MA USA.

### Formation of Gold Nanoparticle Seeds

The final chiral gold nanoparticles used in the present disclosure were grown from gold nanoparticle seeds having the shape of a nanoprism, a nanocube, or an octahedron. These gold nanoparticle seeds were created using the following protocols. The gold nanoprism seeds were created by the following process, 1.6 mL of 0.1 M CTAC was injected into 8 mL of deionized water followed by addition of 75 μL of 10 mM KI and 100.4 μL of sodium tetrachloroaurate solution, which was obtained by mixing HAuCl₄ and NaOH in a 1:1 ratio. Then, 80 μL of 64 mM ascorbic acid was quickly injected to reduce Au³⁺; simultaneously, the color of the solution changed from light yellowish to colorless. Finally, 10 μL of 0.1M NaOH was rapidly injected into the solution to initiate the reduction of Au⁺ for 10 minutes. As the reaction was completed, the color of the solution changed from colorless to blue. The formed gold nanoprisms {111} had an average edge length of 75±3 nm. This procedure is based on that found in Chen, L. et al. High-yield seedless synthesis of triangular gold nanoplates through oxidative etching. Nano Lett. 14, 7201-7206 (2014). The gold nanocube seeds were created by using a two-step process. were synthesized by a two-step process. In a first step, 0.25 mL of 10 mM HAuCl₄ was mixed with 7.5 mL of 0.1 M CTAB. Then, 0.8 mL of 10 mM ice-cold NaBH₄ was quickly injected; the color of the solution changed to dark brown. After rapid stirring for 2 minutes, this reaction solution was placed in a 30° C. water bath for 3 hours. The second step was as follows. First, 1.6 mL of 0.1 M CTAB and 0.2 mL of 10 mM HAuCl₄ were injected into 8 mL of deionized water. Then, 0.95 mL of 0.1 M ascorbic acid was injected; the color of the reaction solution then became colorless. The gold seed solution obtained by the first step reaction was diluted 1:10 in deionized water. Then, 5 μL of the diluted seed solution from the first step was injected into the reaction solution from the second step, thoroughly mixed, and left undisturbed for 15 minutes. This procedure is based on that found in Ahn, H. Y. et al., Extended gold nano-morphology diagram: Synthesis of rhombic dodecahedra using CTAB and ascorbic acid. J. Mater. Chem. C 1, 6861-6868 (2013). The gold octahedron seeds were synthesized as follows: 0.4 mL of PDDA and 0.4 mL of 1 M HCl solution were injected into 20 mL of EG solution in a bottle; the mixture was then stirred at room temperature for 1-2 minutes. Then, 0.02 mL of 0.5 M HAuCl₄ was introduced under stirring; the final concentrations of AuCl₄⁻ ions and PDDA were approximately 0.5 mM and 25 mM, respectively. Finally, the bottle containing the prepared gold precursor solution was sealed and heated in an oil bath at 195° C. for 30 minutes. The protocol follows that found in Lee, E. J. et al., A Facile Polyol Route to Uniform Gold Octahedra with Tailorable Size and Their Optical Properties. ACS Nano 2, 1760-1769 (2008). The formed gold nanoprism seeds, gold nanocube seeds or gold octahedron seeds were then used as seeds to grow the NPs used in the present disclosure. The formed gold nanoprism seeds, gold nanocube seeds and gold octahedron seeds were stored in 1 mM CTAB at a concentration of 0.5 mM.

### Polarized Light Mediated Synthesis of Chiral Gold Nanoparticles

The laser wavelength used in the polarized light mediated synthesis of gold nanoparticles lay within the UV-Vis absorption range of the gold seeds and the circular dichroism of the chiral NP growth. For the chiral NPs synthesized from the gold nanoprisms and gold octahedrons as seeds, the polarized light was used at a wavelength of 594 nm due to the absorption spectra of the seeds. For chiral NPs synthesized from gold nanocubes as seeds, the polarized light was used at a wavelength of 532 nm. When the growth reaction was complete, the reaction solution was centrifuged twice at 1,600 ×g for 1 minute and the pellet was re-suspended in 1 mM CTAB or 5 mM CTAC at a concentration of 50 nM for the formed nanoparticles. The resuspended chiral gold nanoparticles were stable for 3 to 4 weeks in the solutions.

To synthesize L/D-P^{X}NPs, a growth solution was formed by adding 0.8 mL of 10 mM CTAB to 3.95 mL deionized water, followed by the addition of 0.2 mL of 10 mM HAuCl₄. After incubation for 5-10 minutes, 0.475 mL of 40 mM ascorbic acid was injected rapidly into the growth solution. Then, 5 μL of 4 mM L-cysteine-phenylalanine (CYP) or D-cysteine-phenylalanine (CYP)and 50 μL 0.5 mM of gold nanoprism seeds were injected into the growth solution, which was mixed thoroughly. Then, the reaction solution was injected into a quartz cuvette and immediately illuminated with various forms of polarized light (right circularly polarized light, RCP; linear polarized light, LP; left circularly polarized light, LCP) at a wavelength of 594 nm and an intensity of 84 mW/cm² for the indicated period of time, preferably 30 to 40 minutes. In some experiments the time of illumination ranged from 0 to 40 minutes, the best results in terms of increase in g-factor were achieved with an exposure time of 30 to 40 minutes.

To synthesize L/D-P^{X}NP-C, the gold seeds used were the gold nanocubes prepared as described herein. The growth solution was created by adding 1 mL of 0.15 M CTAC and 0.2 mL of 10 mM HAuCl₄ to 3.9 mL deionized water. After incubation for 5-10 minutes, 0.475 mL of 0.1 M ascorbic acid was injected rapidly into the growth solution. Then, 5 μL of 1 mM L/D-CYP and 50 μL of 0.5 mM of gold nanocube seeds were injected into the growth solution and mixed thoroughly. Then, the reaction solution was injected into a quartz cuvette and immediately illuminated at 532 nm with an intensity of 84 mW/cm² under different forms of polarized light (RCP, LP, LCP) for the indicated time period, preferably 30 to 40 minutes. In some experiments the time of illumination ranged from 0 to 40 minutes, the best results in terms of increase in g-factor were achieved with an exposure time of 30 to 40 minutes.

To synthesize L/D-P^{X}NP-O, the gold seeds used were gold octahedron seeds created as described herein. The growth solution was created by adding 1 mL of 0.18 M CTAC and 0.2 mL of 10 mM HAuCl₄ to 3.9 mL deionized water. After incubation for 5-10 minutes, 0.475 mL of 0.3 M ascorbic acid was injected rapidly into the growth solution. Then, 5 μL of 1.2 mM L/D-cysteine-proline (CPR) and 50 μL of 0.5 mM of seeds were injected into the growth solution and mixed thoroughly. The reaction solution was then injected into a quartz cuvette and immediately illuminated at a wavelength of 594 nm and intensity of 84 mW/cm² under different forms of polarized light for the indicated period of time, preferably 30 to 40 minutes. In some experiments the time of illumination ranged from 0 to 40 minutes, the best results in terms of increase in g-factor were achieved with an exposure time of 30 to 40 minutes.

### PEGylation of Nanoparticles

The chiral NPs were modified with mPEG-SH to form PEGylated chiral NPs using the following process: 100 μL of 50 mM mPEG-SH (purchased from Sinopharm Chemical Reagent Co, Ltd. (Shanghai, P.R. China)) was added to 1 mL of the selected 5 nM to 50 nM NP mixture and incubated for 12 hours at 37° C. Then the solution was centrifuged at 1,700 g for 1 minute and the supernatant was discarded. The lower sediment was resuspended in culture medium for in-vitro and in-vivo experiments. Thus, all of the nanoparticles utilized in the studies discussed in this disclosure for both in vitro and in vivo studies were PEGylated nanoparticles as described herein. The PEGylation increased the stability of the chiral nanoparticles in physiologic environments and completely displaced all of the CYP dipeptide from the surface of the chiral nanoparticles used in the experiements described herein. As known to those of skill in the art PEGylation of nanoparticles is commonly used to prevent NP aggregation.

### Calculation Methods

The g-factors, an optical anisotrophy factor, were calculated using the following formula, where the values of circular dichroism were acquired from the circular dichroism spectra and the light absorbance values were obtained from the UV-Vis spectra. The notation +/−before g-factor indicates that it may refer to left/right polarization rotation specifically. If no sign is used in front of the g-factor, this value refers to an absolute value of this parameter.

g-factor=circular dichroism(mdeg,from circular dichroism spectrometer)/(32980×absorbance value(from UV−Vis spectrometer))

### Calculation of the Number of Peptides on a Single Nanoparticle

The number of surface ligand cysteine-phenylalanine (CYP) dipeptides per prepared nanoparticle, the number of surface ligand cysteine-phenylalanine (CYP) dipeptides per nanoparticle after PEGylation and the number of surface ligand cysteine-phenylalanine (CYP) dipeptides per prepared nanoparticle following treatment of the nanoparticle with DTT were all determined. To prepared nanoparticles mPEG-SH and dithiothreitol (DTT) were added, the final concentrations were 5 mM and 50 mM, respectively, to 1 mL of the prepared nanoparticles to displace the CYP from the nanoparticles. After incubation for 12 hours, the supernatant was discarded following centrifugation at 1,700 g for 1 minute and the sediment was re-suspended in 1 mL of 1 mM CTAB. To quantify the amount of CYP molecules on the surface of the three populations of the chiral NPs described herein NaBH₄ was added to each nanoparticle solution, the reductive desorption reaction of adsorbed thiolate molecules (Au-SR) started immediately. The final concentration of NaBH₄ was 50 mM and the final volume of the solution was fixed to 1 mL. After 5 min of incubation, the NPs were centrifuged again and clear supernatant solutions containing the released CYP molecules were collected. Quantification of CYP on the chiral NPs was carried out using liquid chromatography-mass spectrometry. The number of CYP molecules on a single NP surface was calculated to be 1.39×10⁴. After PEGylation modification or treatment with DTT all of the CYP dipeptides were displaced from the surface of the chiral NPs. All the chiral NPs used in the experimental studies herein were PEGylated as described above.

### Quantification of Nanoparticle Chirality

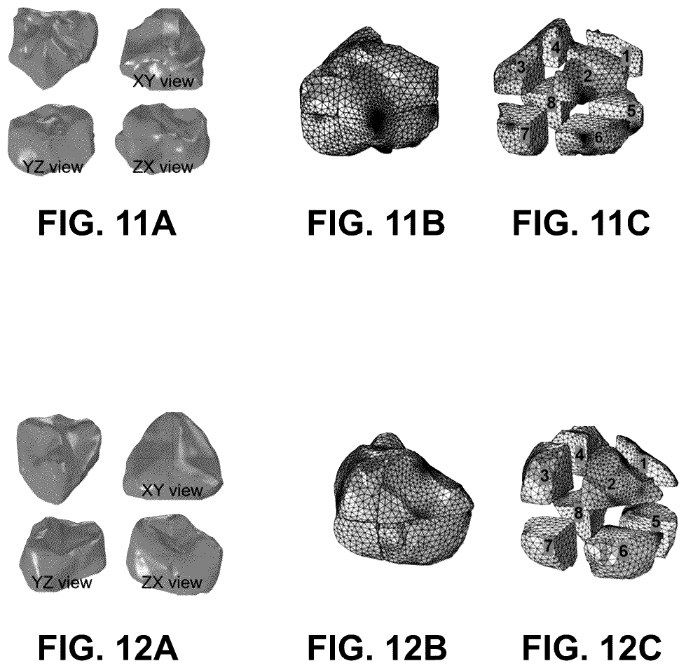

Quantification of chirality of NPs was carried out as follows. First mass point groups were collected from electron tomography. A chiral object or molecule may be uniquely represented by a generalized density, p(r), which, for a molecule consisting of point atoms, could be the sum of delta functions. Instead of using coordinates having actual mass point of nuclei or atom, we calculated a coordinate of mass centers from partitions of nanoparticle geometry to simplify spatial information of mass of entire geometry. We imported electron tomograms of three different groups of enantiomers into a Cartesian coordinate system having an origin at the center of mass referred to as the primary center of mass. The first moment of inertia principal direction of each NP structure is aligned with z axis while the second and third moment of inertia principal directions are aligned with x and y axes, respectively. With the eight octants of the coordinate system, the particles were sectioned into pieces, see FIGS. 11B-11C and 12B-12C, and coordinates from their secondary center of masses have been calculated.

The Hausdorff chirality measure (HCM) calculation was computed using MATLAB software according to the published reference. Johnson, P. B. et al., Optical Constants of the Noble Metals. Phys. Rev. B 6, 4370-4379 (1972). The implementation of HCM calculations adapted for nanoscale structures followed the general protocol described by J. Kim et al. (J-Y. Kim, J.Yeom, G.Zhao, H.Calcaterra, J. Munn, P. Zhang, N. A. Kotov, Assembly of Gold Nanoparticles into Chiral Superstructures Driven by Circularly Polarized Light, Journal of American Chemical Society, 2019, 141, 30, 11739-11744). By rotating and translating one enantiomer with respect to the other, one can find the minimal value of Hausdorff's distances corresponding to the optimal overlap(s). Hausdorff chirality measure Q, HCM(Q), then can be expressed as the following:

$$HCM ( Q ) = h ( min ) ( Q , {Q}^{′} ) / d ( Q )$$

where Q and Q′ denote two enantiomorphic sets of points; d (Q) is the largest distance between any two points of Q. The calculation of HCM(Q) requires the analysis of many different superimpositions of Q and its mirror image Q′. The value of H(Q) depends on six variables; u, v, and w, describe translations of Q′ along x, y, and z axes, respectively, and α, β, and γ, define rotations around those axes. Enantiomorphs (Q′) of original point set for each particle, (Q), were obtained by operating inversion. Hausdorff distances of these two enantiomorphic sets of points groups were evaluated with Broyden-Fletcher-Goldfarb-Shano (BFGS) numerical procedure as function of u, v, w, α, β and γ, where α, β, and γ were defined every 1° rotation angle step around the x, y, and z axes.

The chirality measure approach through Hausdorff distance described above cannot provide the sign for the chiral structures, hence we also used the Osipov-Pickup-Dunmeur chirality index (OPD). Johnson, P. B. et al., Optical Constants of the Noble Metals. Phys. Rev. B 6, 4370-4379 (1972). Osipov et al. suggested a different method to quantify molecular chirality with signs using intrinsic molecular chirality tensor based on only on nuclear positions. Here, we have obtained the chiral index by calculating summation of contributions of all sets of four points from nine coordinates (primary and secondary center of masses collected by method described above) using MATLAB code. The tensor gives rise to two universal chirality indices; the first giving information about absolute chirality, and the second about the anisotropy chirality, that is, the degree of chirality in different spatial directions. The pseudoscalar behavior of chiral molecule can be described by the gyration tensor G,

$$G = \int {dr}_{1} {dr}_{2} {dr}_{3} {dr}_{4} ρ ( {r}_{1} ) ρ ( {r}_{2} ) ρ ( {r}_{3} ) ρ ( {r}_{4} ) \times \frac{[ ( {r}_{12} \times {r}_{34} ) ⊗ {r}_{14} ] ( {r}_{12} \cdot {r}_{23} ) ( {r}_{23} \cdot {r}_{34} )}{{( {r}_{12} {r}_{23} {r}_{34} )}^{n} {r}_{14}^{m}}$$

where rᵢⱼ=rᵢ−rⱼ and \hat{r}ᵢⱼ=rᵢⱼ/rᵢⱼ and n and m are arbitrary integers and ρ stands for the density of concentration. A chiral index, G₀, is a summation of contributions of all sets of four atoms in a numerical evaluation given by

$${G}_{0} = \frac{1}{3} [ {\sum }_{\begin{matrix}all permutations of \\ i , j , k , l = 1 … N\end{matrix}} {w}_{i} {w}_{j} {w}_{k} {w}_{l} \times \frac{[ ( {r}_{ij} \times {r}_{kl} ) \cdot {r}_{il} ] ( {r}_{il} \cdot {r}_{jk} ) ( {r}_{jk} \cdot {r}_{kl} )}{{( {r}_{ij} {r}_{jk} {r}_{kl} )}^{n} {r}_{il}^{m}} ]$$

where w is atomic weight=1 for all molecules considered here to provide a measure of steric chirality.

### Cells and Cell Cultures

Mouse bone-marrow-derived macrophages (BMMs) were separated from C57BL/6 mice and cultured in PRMI 1640 medium plus 10% fetal bovine serum (FBS) with 100 ng/mL of macrophage colony-stimulating factor (Biolegend) for 7 days. Mouse bone-marrow-derived dendritic cells (BMDCs) were separated from wild type or NLRP3 knockout (NLRP3^{-/-}) C57BL/6 mice and cultured in PRMI 1640 medium plus 10% FBS with 10 ng/mL of granulocyte macrophage colony-stimulating factor (Biolegend) for 7 days. Human BMDCs, obtained from Procell Life Science &Technology Co., Ltd., were cultured in PRMI 1640 medium plus 10% FBS for 7 days. The nucleotide-binding oligomerization domain-like receptors, or NOD-like receptors (NLRs), family pyrin domain containing 3 is known as NLRP3. It is a protein expressed mainly in macrophages and is a component of the inflammasome.

### Apoptosis Assays

Mouse BMMs and BMDCs or human BMDCs were seeded into 6-well plates at an initial density of 10⁶ cells per well and incubated with different chiral nanostructure concentrations of 0, 0.5, 1, 2 and 4 nM. After 12 hours, the cells were harvested and stained with Annexin V and propidium iodide (PI) (Beyotime, C1052) for 15 minutes in the dark. Results were analyzed by CytExpert.

### Toxicity In-Vivo

All animal experiments complied with institutional ethical guidelines and Committee on Animal Welfare of Jiangnan University. The tail veins of C57BL/6 mice were injected with L-P⁺NPs and D-P⁻NPs, 2 mg. On days 1, 3, 5, 7, and 15, the mice were euthanized and the liver and kidney were excised for hematoxylin and eosin staining. Blood samples were collected on days 1, 3, 5, 7, and 15 by eyeball extraction and used to test liver and kidney function.

### In-Vitro Cellular Uptake

Immune cells were seeded into 6-well plates. The cells (1×10⁶ cells per well) were incubated with nanomaterials (L-P⁺NPs, D-P⁻NPs, L-P⁺NPs-DTT and D-P⁻NPs-DTT) at a concentration of 2 nM for different times periods. The L-P⁺NPs-DTT and D-P⁻NPs-DTT are L-P⁺NPs and D-P⁻NPs that have been treated with DTT to remove any dipeptide surface ligands. Then, the culture medium was discarded. The cells were then collected and washed three times with 1 x Dulbecco's phosphate-buffered saline (DPBS; Life Technologies) and re-suspended in 1 mL of DPBS. The uptake level was then expressed as the UV-Vis absorbance and the circular dichroism signal.

### Confocal Microscopy Imaging

Intracellular transport of NPs was observed by confocal microscopy. The cells were seeded in a 35 mm Petri dish and cultured for 24 hours to achieve a density of 10⁴ cells per plate. Then, the cells were incubated with nanomaterials (L-P⁺NPs, D-P⁻NPs, L-P⁺NPs-DTT, D-P⁻NPs-DTT, and Cy3-PEG-L-P⁺NPs, 2 nM) in culture medium for different times. Then, the culture medium was discarded, the cells were washed three times with DPBS, fixed with 4% paraformaldehyde for 10 minutes, stained with 4′,6-diamidino-2-phenylindole (DAPI) (Beyotime, C1005) and observed by laser scanning confocal microscopy (Leica TCS SP8). The gray values of the white dots were then analyzed by LAS AF Lite software. To estimate the ability of recruitment of dynamin, the cells were stained with Cy5-labeled CD97 antibody, Cy5-labled EMR1 dynamin antibody (Cell signaling technology, 2342S), or clathrin antibody (Cell signaling technology, 4796S), and secondary antibody, respectively (Thermofisher, 84541).

### Isothermal Titration Calorimetry (ITC) Studies

The chiral nanocrystals (100 μM) were suspended in DPBS and injected into the CD97 or EMR1 (10 μM) with 1.96 μL per injection (injection interval is 300 seconds, 25 injections in total). The thermodynamic effects were measured by a Nano ITC Low Volume (TA Instrument, USA). The stirring rate was 300 rpm during the measurements. The data were analyzed by the original NanoAnalyze software.

### In-Vitro Activation and Cytokine Secretion Measurements

Mouse BMMs and BMDCs were seeded into 6-well plates (10⁶ cells/well) and cultured with 2 μg/mL monophosphoryl lipid A (MPL), 20 μg/mL ovalbumin (OVA) and L-NPs, L-P⁺¹⁰NPs, L-P⁺¹⁵NPs, L-P⁺²⁰NPs, L-P⁺²⁵NPs, L-P⁺NPs, nanoprism seeds, D-NPs, D-P⁻¹⁰NPs, D-P⁻¹⁵NPs, D-P⁻²⁰NPs, D-P⁻²⁵NPs, D-P⁻NPs, PBS, mPEG-SH (Mw=2,000), L-CYP, D-CYP, NP, NP-D-CYP, NP-L-CYP, NS-L-CYP, NS-D-CYP, L-P⁺NPs-DTT, and D-P⁺NPs-DTT (2 nM, respectively), for 12 hours. Afterwards, the supernatant was collected and Interleukin-12 (IL-12) (BD, 555256) and Interleukin-1β (IL-1β) (JKBio Shanghai, JLC3580) production was estimated using an enzyme-linked immunosorbent assay (ELISA) kit. Cells were then harvested and stained using CD86 monoclonal antibody (GL1), CD40 monoclonal antibody (1C10), CD80 monoclonal antibody (16-10A1), OVA257-264 (SIINFEKL) peptide bound to H-2Kb monoclonal antibody (25-D1.16), MHC Class II (I-A/I-E) monoclonal antibody (M5/114), or anti-TNF-α (TN3-19.12). Flow cytometry data were analyzed by FlowJo10.3 and GraphPad prism software.

### Western Blotting Analysis

For the western blotting analysis, mouse BMDCs (1.0×10⁶) cultured in cell medium were collected, and their proteins were extracted with RIPA Lysis Buffer IV (Beyotime). The protein lysates were separated with sodium dodecyl sulphate-polyacrylamide gel electrophoresis (SDS-PAGE), transferred onto polyvinylidene difluoride (PVDF) membranes, and the blot was processed according to the protocol of the manufacturer of the kit used (Sangon Biotech Co., Ltd). The PVDF membranes were incubated with a primary antibody (diluted 1:1,000) directed against clathrin, dynamin NLRP3, IL-1β, pro-caspase-1, or caspase-1, and then with a horseradish-peroxidase-conjugated secondary antibody (1:500 dilution), respectively, and R-actin was used as a loading control.

Human BMDC (1.0×10⁶) cultured in the specified cell medium were collected and their proteins were extracted with RIPA Lysis Buffer IV (Beyotime). The protein lysates were separated with SDS-PAGE, transferred onto polyvinylidene difluoride (PVDF) membranes, and the blot was processed according to the manufacturer protocol of the western blotting analysis kit from Sangon Biotech Co., Ltd. The PVDF membranes were incubated with a primary antibody (diluted 1:1,000) directed against CD97, EMR1, and then with a horseradish-peroxidase-conjugated secondary antibody (1:500 dilution), respectively. β-actin was used as a loading control.

### Inflammasome Activation

Mouse BMDCs collected from wild type or NLRP3 knockout (NLRP3^{-/-}) C57BL/6 mice were incubated with 2 μg/mL MPL, 20 μg/mL OVA, and 2 nM L-NPs, L-P⁺¹⁰NPs, L-P⁺¹⁵NPs, L-P⁺²⁰NPs, L-P⁺²⁵NPs, L-P⁺NPs, nanoprism seeds, D-NPs, D-P⁻¹⁰NPs, D-P⁻¹NPs, D-P⁻²⁰NPs, D-P⁻²⁵NP,s D-P⁻NPs, PBS, L-CYP, D-CYP, NP (these are nanoparticles synthesized without polarized light or chiral ligands, see Table 1), NP-D-CYP, NP-L-CYP, NS-L-CYP and NS-D-CYP (2 nM), for 12 hours. The IL-1β concentration in the culture medium was measured with an ELISA kit. Cells were treated by NLRP3 antibody and Alexa Fluor 488-conjugated goat anti rabbit IgG secondary antibody. Then, the NLRP3 expression was measured by confocal imaging, flow cytometry, and RT-PCR. To explore the signal pathway of the inflammasome activity by chiral NPs, mouse BMDCs were pre-treated with MCC950, a known potent small-molecule inhibitor of the NLRP3 pathway (10 M), chlorpromazine (50 μM), cytochalasin D (2 μM), NAC (N-acetyl-L-cysteine 5 mM), amiodarone (40 μM), KCl (130 mM), dynasore (80 μM), nocodazole (10 μM), or CA-074-Me (5 μM) for 2 hours and then co-cultured with L-P⁺NPs (2 nM) or D-P⁺NPs (2 nM) for 12 hours.

Wild type or NLRP3 knockout (NLRP3^{-/-}) C57BL/6 mice were subcutaneously immunized with different chiral NPs (2 mg), MPL (10 μg), and OVA (50 μg), respectively. After 36 hours, the draining lymph nodes (dLNs) were collected. The expression of NLRP3 in mouse BMDCs was analyzed by flow cytometry.

### In-Vivo Immune Responses

Wild type or NLRP3 knockout (NLRP3^{-/-}) C57BL/6 mice were subcutaneously immunized with nanoprism seeds (2 mg)+monophosphoryl lipid A (MPL) (10 μg)+ovalbumin (OVA) (50 μg), L-NP (2 mg)+MPL (10 μg)+OVA (50 μg), L-P⁺¹⁰NPs (2 mg) +MPL (10 μg)+OVA (50 μg), L-P⁺¹⁵NPs (2 mg)+MPL (10 μg)+OVA (50 μg), L-P⁺²⁰NPs (2 mg)+MPL (10 μg)+OVA (50 μg), L-P⁺²⁵NPs (2 mg)+MPL (10 μg)+OVA (50 μg), L-P⁺NPs (2 mg)+MPL (10 μg)+OVA (50 μg), D-NP (2 mg)+MPL (10 μg)+OVA (50 μg), D-P⁻¹⁰NPs (2 mg)+MPL (10 μg)+OVA (50 μg), D-P⁻¹⁵NPs (2 mg)+MPL (10 μg)+OVA (50 μg), D-P⁻²⁰NPs (2 mg)+MPL (10 μg)+OVA (50 μg), D-P⁻²⁵NPs (2 mg)+MPL (10 μg)+OVA (50 μg), D-P⁻NPs (2 mg)+MPL (10 μg)+OVA (50 μg), PBS +MPL (10 μg)+OVA (50 μg), mPEG-SH (10 mg)+MPL (10 μg)+OVA (50 μg), L-CYP (10 μg)+MPL (10 μg)+OVA (50 μg), D-CYP (10 μg)+MPL (10 μg)+OVA (50 μg), NP (2 mg)+MPL (10 μg)+OVA (50 μg), L-P⁺NPs-DTT (2 mg, these are NPs treated with DTT to remove any surface ligand dipeptides)+MPL (10 μg)+OVA (50 μg), D-P⁻NPs-DTT (2 mg, these are NPs treated with DTT to remove any surface ligand dipeptides)+MPL (10 μg)+OVA (50 μg), NS-L-CYP (2 mg)+MPL (10 μg)+OVA (50 μg), NS-D-CYP (2 mg)+MPL (10 μg)+OVA (50 μg), NP-L-CYP (2 mg)+MPL (10 μg)+OVA (50 g), or NP-D-CYP (2 mg)+MPL (10 μg)+OVA (50 μg).

To evaluate the maturation of mouse BMDCs in-vivo, mice were euthanized and the inguinal lymph nodes collected to prepare single cell suspensions 36 hours after immunization. Cells were stained using the following antibodies: FITC CD1 1c monoclonal antibody (N418), PerCP-eFluor710 CD40 monoclonal antibody (1C10), PE CD80 monoclonal antibody (16-10A1), APC CD86 monoclonal antibody (GL1), OVA257-264 (SIINFEKL) peptide bound to H-2Kb monoclonal antibody (25-D1.16), MHC Class II (I-A/I-E) monoclonal antibody (M5/114) (eBioscience, Thermo Fisher Scientific).

To analyze immune responses, mice were euthanized at 0 and 14 days and splenocytes were harvested 7 days post the last immunization. Splenocytes were stimulated overnight with OVA. Cell Activation Cocktail with Brefeldin A (Biolegend, 423304) was added to the cell culture in the final 4 hours. Splenocytes were stained by FITC CD3e Monoclonal Antibody (145-2C11), APC CD8a Monoclonal Antibody (53-6.7), PE-Cy7 Rat Anti-mouse TNF-α (MP6-XT22), and PerCP-Cyanine5.5 IFN-γ Monoclonal Antibody (XMG1.2). For immune memory evaluation, splenocytes were co-stained by FITC CD3e Monoclonal Antibody (145-2C11), APC CD8a Monoclonal Antibody (53-6.7), PE CD44 Monoclonal Antibody (IM7), and PE-Cyanine7 CD62L Monoclonal Antibody (MEL-14). All the antibodies were obtained from eBioscience, Thermo Fisher unless otherwise indicated. The antibodies dilution for flow cytometry staining was performed according to the manufacturer's handbook.

### Influenza Vaccination

C57BL/6 mice were immunized at 0 and 14 days with indicated formulations including H9N2 influenza vaccine (10⁸ ELD₅₀/0.1 mL, 60 μL), MPL (10 μg), L-P⁺NP (2 mg)+H9N2+MPL, D-P⁻NPs (2 mg)+H9N2+MPL, 50 μL Alum (Thermo Fisher Scientific, 77161)+H9N2+MPL, L-P⁺NPs-DTT (2 mg)+H9N2+MPL, D-P⁻NPs-DTT (2 mg)+H9N2+MPL, NS-L-CYP (2 mg)+H9N2+MPL, NS-D-CYP (2 mg)+H9N2+MPL. Then 7 days after immunization, mice were euthanized and splenocytes were harvested and stimulated with the influenza virus for 12 hours. Cell Activation Cocktail with Brefeldin A (Biolegend, 423304) was added to the cell culture in the final 4 hours. Splenocytes were stained by FITC CD3e Monoclonal Antibody (145-2C11), APC CD8a Monoclonal Antibody (53-6.7), PE-Cy7 Rat Anti-mouse TNF-α (MP6-XT22), PerCP-Cyanine5.5 IFN-γ Monoclonal Antibody (XMG1.2), and PE IL-4 Monoclonal Antibody (11B11). The antibodies dilution for flow cytometry staining was performed according to the manufacturer's handbook. The immunized mice were challenged with H9N2 influenza virus 14 days after the last immunization. Then 21 days after being challenged, mice were euthanized and lungs were harvested for hematoxylin and eosin (H&E) staining.

### Antibody Titer Test

C57BL/6 mice were immunized with OVA (50 μg) or H9N2 influenza vaccine (10⁸ ELD₅₀/0.1 mL, 60 μL) with the indicated adjuvants, including MPL, 50 μL Alum (Thermo Fisher Scientific, 77161)+10 μg MPL, 2 mg D-P⁻NP+10 μg MPL, 2 mg L-P⁺NP+10 μg MPL for three times (day 1, day 14, and day 56). The OVA or H9N2 specific Serum IgG titers were collected from day 0 and over 91 days and measured by ELISA kit according to the protocol (JingMei biotechnology).

### FDTD Simulations

Optical properties and growth mechanisms for Au chiral nanoparticles were simulated using finite-difference time-domain (FDTD) software (Lumerical FDTD Solutions). The Au chiral nanoparticles were illuminated using a normally incident right-handed circularly polarized (RCP) and left-handed circularly polarized (LCP) plane wave propagating in the z direction. We used the ‘two sources in one simulation method’ for making CPL. The phase of the x polarized plane wave source was set to 0 and the phase of the y polarized plane wave source was set to positive or negative 90 degrees. Positive and negative 90 degrees of phase were defined as LCP and RCP, representing counterclockwise rotation and clockwise rotation along the propagation axis, respectively. The optical properties of gold were from Johnson and Christy. Johnson, P. B. & Christy, R. W. Optical Constants of the Noble Metals. Phys. Rev. B 6, 4370-4379 (1972). The chiral structure was excited by a source with a wavelength range from 200 to 1000 nm, propagating along the negative z direction. A simulation box size of 0.5 μm×0.5 μm×1.2 μm was used. Perfectly matched layer (PML) absorbing boundaries were applied for the top and bottom x-y planes and periodic boundaries were applied for the front and back x-z planes and the left and right y-z planes. The geometries of chiral gold nanoparticles were reconstructed using numerical computing software (MATLAB) and 3D graphic software packages (3D Max 2017, Autodesk). Circular dichroism in the simulations was defined as CD=|A_{L}−A_{R}|, where A_{L} and A_{R}represent the absorbance of LCP and RCP photons, respectively.

### Electrodynamics Calculations of Scattering, Extinction, Absorption and Circular Dichroism

The simulation of the interaction between electromagnetic radiation and the gold nanoparticles by means of classical electrodynamics requires a number of choices regarding the model to be made beforehand, as we now discuss. First, we define the radiation electric using complex exponentials,

$$\tilde{E} ( r , ω , t ) = {\hat{E}}_{0} ( r , ω ) {e}^{- i ω t} {e}^{i φ} = {E}_{0} ( r , ω ) {e}^{- i ω t} \qquad ( 1 )$$

where \tilde{E} is the time-dependent electric field, Ê₀ is the real amplitude, E₀ is the complex amplitude, φ is the absolute phase, ω is the angular frequency, and t is the time. The frequency may be related with wavelength, λ, by

$$ω = \frac{2 π c}{λ} = {k}_{0} c \qquad ( 2 )$$

where the wavevector k₀=|k₀| is wavenumber of light in vacuum and the speed of light in vacuum is given by

$$c = \frac{1}{\sqrt{{ε}_{0} {μ}_{0}}} . \qquad ( 3 )$$

The electric field, E₀, can be represented by a plane wave, and its complex amplitude is equal to:

$${E}_{0} ( r , ω ) = {\hat{E}}_{0} {e}^{- i {k}_{0} \cdot r} . \qquad ( 4 )$$

In the present work, we have defined circularly polarized light as propagating along the z direction (φ=0), yielding the following field components:

$${E}_{0 , x} = {E}_{x 0} {e}^{( i {k}_{z} z )} \qquad ( 5 )$$

$${E}_{0 , y} = {E}_{y 0} {e}^{( i {k}_{z} z )} {e}^{( i ϕ )} \qquad ( 6 )$$

$${E}_{z} = 0. \qquad ( 7 )$$

A priori, Eₓ₀=E_{y0}=1 and k_{z}=+1, indicating that the electric field moves from negative to positive values of z as it moves towards the nanoparticle, and ϕ=π/2 for LCP and ϕ=−π/2 for RCP.

Bearing these definitions in mind, the Maxwell relations can be expressed as

$$∇ \cdot E ( r , ω ) = \frac{4 π}{{ε}_{e n v}} ρ ( r , ω ) \qquad ( 8 )$$

$$∇ \times E ( r , ω ) = i {k}_{0} B ( r , ω ) \qquad ( 9 )$$

$$∇ \cdot B ( r , ω ) = 0 \qquad ( 10 )$$

$$∇ \times B ( r , ω ) = - i {k}_{0} {ε}_{e n v} E ( r , ω ) - \frac{4 π}{c} j ( r , ω ) \qquad ( 11 )$$

where ρ is the charge density, j is the current density associated with the nanostructure in an environment of permittivity εₑₙᵥ, and B is the time-dependent magnetic field.

The Maxwell's equations establish the relations between the fields generated by currents and charges in matter, but without defining how these charges and currents have been achieved, requiring a self-consistent procedure to link the electrostatic properties of matter and the incoming fields. This procedure leads to a wave equation (See Griffiths, D. J. Electromagnetic Waves. in Introduction to Electrodynamics 364-411 (John Wiley & Sons, Inc., 1989). doi:10.1002/0471206466.chl.),

$$( Δ + {k}^{2} ) E ( r , ω ) = - \frac{1}{{ε}_{e n v}} ∇ ∇ P ( r , ω ) + {ω}^{2} {μ}_{0} P ( r , ω ) . \qquad ( 12 )$$

Assuming a non-magnetic medium with μᵣ=μₑₙᵥ=1, then n=\sqrt{μᵣεr}=\sqrt{εᵣ} and the wave equation reduces to

$$( Δ + {k}^{2} ) E ( r , ω ) = - \frac{1}{{ε}_{e n v}} ( {k}^{2} + ∇ ∇ ) P ( r , ω ) \qquad ( 13 )$$

where P=Xₑ·E is the electric polarization vector and k=\sqrt{εₑₙᵥ}k₀ is the wavenumber in the environment medium.

This inhomogeneous linear partial differential equation may be conveniently solved using Green's functions, yielding the only physically acceptable solution (See Economou, E. N. Time-Independent Green's Functions. in Green's Functions in Quantum Physics vol. 7 3-19 (2006)) as

$${G}_{0} ( r , {r}^{′} ) = \frac{1}{4 π} . \frac{{e}^{\pm i k \vert r - {r}^{′} \vert}}{\vert r - {r}^{′} \vert} = \frac{{e}^{\pm i k R}}{4 π R} \qquad ( 14 )$$

where R=|R|=|r−r′|, r is the electromagnetic radiation source point and r′ are all possible observation points. Nonetheless, the scalar Green's functions are not sufficient to actually solve the wave equation, being necessary to extend the scalar Green's functions into the corresponding vector field, i.e., the Green's functions must become the tensor form of the Dyadic Green's functions. See Novotny, L. & Hecht, B. Theoretical foundations. in Principles of Nano-Optics vol. 66 12-44 (Cambridge University Press, 2012).

Equation 14 has a singularity at r=r′, which we have removed before discretizing space using a hexagonal regular mesh (See Girard, C., Dujardin, E., Baffou, G. & Quidant, R. Shaping and manipulation of light fields with bottom-up plasmonic structures. New J. Phys. 10, 105016 (2008)), yielding

$${G}_{0}^{E E} ( {r}_{i} , {r}_{i} ) = - \frac{4 π \sqrt{2}}{3 {ε}_{e n v} {d}^{3}} I \qquad ( 15 )$$

where d is the edge length of the volume element used for the space discretization and I is the unit tensor. Deriving the Lippmann-Schwinger equation and using the Green's function concepts, gives us a vectorial Lippmann-Schwinger equation,

$$E ( r , ω ) = {E}_{0} ( r , ω ) + \int {G}_{0}^{E E} ( r , {r}^{′} , ω ) \cdot {χ}_{e} E ( {r}^{′} , ω ) {dr}^{′} . \qquad ( 16 )$$

The integration is performed over the material volume, assuming that it has a nonzero electric susceptibility (Xₑ≠0), but since equation 16 does not have an analytical solution for arbitrary shapes, it was rewritten for a cubic mesh with V_{cell}=d³ as

$$E ( {r}_{i} , ω ) = {E}_{0} ( {r}_{i} , ω ) + {\sum }_{j = 1}^{N} {G}_{0}^{E E} ( {r}_{i} , {r}_{j} , ω ) \cdot {χ}_{e} E ( {r}_{j} , ω ) {V}_{cell} \qquad ( 17 )$$

where E₀(r,ω) is the incident electric field, E(r,ω) is the total field, XₑE(rⱼ,ω) is the fluctuating dipole moment in the discrete volume element j induced by the local electric field E(rⱼ,ω) and N is number of volume elements comprising the overall volume.

Equation 17 is solved for the field inside the object, while the field outside it is deduced from the self-consistent field inside the material, with each volume element being self-consistently modified by the presence of the others volume elements.

The solution of the vectorial Lippmann-Schwinger equation requires the analytical solution for the Dyadic Green's function G₀^{EE}(r, r′,ω)

$${G}_{0}^{E E} ( r , {r}^{′} , ω ) = \frac{1}{{ε}_{e n v}} ( {k}^{2} I + ∇ ∇ ) {G}_{0} ( r , {r}^{′} , ω ) \qquad ( 18 )$$

$${G}_{0}^{E E} ( r , {r}^{′} , ω ) = \frac{{e}^{i k R}}{{ε}_{e n v}} ( - {k}^{2} {T}_{1} ( R ) - i k {T}_{2} ( R ) + {T}_{3} ( R ) ) \qquad ( 19 )$$

where the tensor Tᵢ(R) elements are defined as

$${T}_{1} ( R ) = \frac{R R - I {R}^{2}}{{R}^{3}} \qquad ( 20 )$$

$${T}_{2} ( R ) = \frac{3 RR - {IR}^{2}}{{R}^{4}} \qquad ( 21 )$$

$${T}_{3} ( R ) = \frac{3 RR - I {R}^{2}}{{R}^{5}} . \qquad ( 22 )$$

And G₀(r, r′,ω) is the scalar Greens's function, RR is the dot product of tensor R. These tensor elements describe either the far-field properties (T₁(R)) or the near-field (T₂(R) and T₃(R)).

Once the iterative self-consistent calculation converges, the extinction cross section may be defined from the nearfield as

$${σ}_{e x t} = \frac{4 π {k}_{0}}{\vert {E}_{0} {\vert}^{2}} {\sum }_{i = 1}^{{N}_{c e l l s}} Im ( {E}_{0 , i}^{*} \cdot {P}_{i} ) . \qquad ( 23 )$$

And the absorption cross section as

$${σ}_{a b s} = \frac{4 π {k}_{0}}{\vert {E}_{0} {\vert}^{2}} {\sum }_{i = 1}^{{N}_{c e l l s}} Im ( {P}_{i} \cdot {E}_{i}^{*} - \frac{2}{3} {{k}_{0}}^{3} \vert {P}_{i} {\vert}^{2} ) . \qquad ( 24 )$$

The total scattering cross section of the material is then σ_{scat}=σₑₓₜ−σ_{abs}, being necessary to repeat the calculation for LCP and RCP to derive the circular dichroism for the overall scattering cross section and its components as the difference σ_{LCP}−σ_{RCP}.

The optical extinction inside any volume element amounts to a heat dissipation, which may be summed up to yield the total dissipated heat Q(a) as

$$Q ( ω ) = 4 π {k}_{0} {P}_{e} {P}_{l} {\sum }_{i = 1}^{{N}_{c e l l s}} Im ( {∈}_{i} ) \vert {E}_{i} {\vert}^{2} . \qquad ( 25 )$$

The numerical factor Pₑ=100 was used to convert the power to nW and Pₗ=0.00000084 mW/μm²=84 mW/cm² is the irradiance of the incident laser power.

Equation 25 establishes a relation between the imaginary part of the complex permittivity and the electric field intensity in the volume element. Although heat itself cannot be regarded as a chiral property, its distribution over the mesh describing the material should be chiral if the shape of the nanostructure is chiral.

The shape of the model structure meant to be used for electrodynamics calculations was extracted by the photogrammetry technique used to construct a surface from the crystal tomography images, converting a set of images into a set of three-dimensional (3D) points that form a closed model surface with the same shape observed experimentally for the gold NP. Python Photogrammetry Toolbox (See Abraham, M. J. et al. GROMACS: High performance molecular simulations through multi-level parallelism from laptops to supercomputers. SoftwareX 1-2, 19-25 (2015)) was used, along with the software Bundler (See Heinz, H., Vaia, R. A., Farmer, B. L. & Naik, R. R. Accurate Simulation of Surfaces and Interfaces of Face-Centered Cubic Metals Using 12-6 and 9-6 Lennard-Jones Potentials. J. Phys. Chem. C 112, 17281-17290 (2008).) for calibration and the CMVS/PMVS software to perform the dense reconstruction.

This preliminary processing yields 3D points without structuration, requiring that further processing should be done using MeshLab (See Bakan, A., Meireles, L. M. & Bahar, I. ProDy: Protein Dynamics Inferred from Theory and Experiments. Bioinformatics 27, 1575-1577 (2011)) to reconstruct the surface as a structured mesh, whose mesh points were converted into a watertight surface using the Screened Poisson Surface Reconstruction. See Wiecha, P. R. pyGDM-A python toolkit for full-field electro-dynamical simulations and evolutionary optimization of nanostructures. Comput. Phys. Commun. 233, 167-192 (2018).

The optimized reconstructed surface was superposed on a grid containing 343000 randomly distributed points and the generalized winding numbers method (See Jacobson, A., Kavan, L. & Sorkine-Hornung, O. Robust inside-outside segmentation using generalized winding numbers. ACM Trans. Graph. 32, 33 (2013)) to determine that 166356 points were inside the reconstructed surface. These randomly distributed points were used to construct a cubic grid using a naive method resulting in the final cubic grid with 4894 points that was used for electrodynamics calculations.

The grid was scaled up to achieve an overall size of 110 nm, 115 nm, and 95 nm, in the x-, y-, and z-directions, respectively. This size is comparable to the experimentally determined size of the NP, with cubic cells inside the surface having 5 nm of edge length and yielding a total of 4894 dipoles. The dyadic Green's functions were solved for this grid using the frequency-dependent refractive index of gold and considering the model NP surrounded by water (nₑₙᵥ=1.33). See Johnson, P. B. & Christy, R. W. Optical Constants of the Noble Metals. Phys. Rev. B 6, 4370-4379 (1972). The power of the light source was set to 84 mW/cm² for all calculations. The results are averaged over 64 rotations of the NP following Euler angles of 90, 180, or 270 degrees around either x-, y-, and z-axes.

TABLE 1
A list of abbreviations to nanoparticles employed in this disclosure.

| | | Reaction | | | | |
|---|---|---|---|---|---|---|
| Abbreviation | Polarized light | time | | Seed | Chiral ligand | g-factor |
| L-P⁺NP | Left circularly | 30 | min | Gold | L-cysteine- | 0.44 |
| | polarized light | | | nanoprism | phenylalanine | |
| L-P⁻NP | Right circularly | | | | | 0.25 |
| | polarized light | | | | | |
| L-P⁰NP | Linearly | | | | | 0.32 |
| | polarized light | | | | | |
| D-P⁺NP | Left circularly | | | | D-cysteine- | 0.25 |
| | polarized light | | | | phenylalanine | |
| D-P⁻NP | Right circularly | | | | | 0.42 |
| | polarized light | | | | | |
| D-P⁰NP | Linearly | | | | | 0.33 |
| | polarized light | | | | | |
| D-P⁻¹⁰NP | Right circularly | 10 | min | | D-cysteine- | 0.16 |
| | polarized light | | | | phenylalanine | |
| L-P⁺¹⁰NP | Left circularly | | | | L-cysteine- | 0.16 |
| | polarized light | | | | phenylalanine | |
| D-P⁻¹⁵NP | Right circularly | 15 | min | | D-cysteine- | 0.24 |
| | polarized light | | | | phenylalanine | |
| L-P⁺¹⁵NP | Left circularly | | | | L-cysteine- | 0.25 |
| | polarized light | | | | phenylalanine | |
| D-P⁻²⁰NP | Right circularly | 20 | min | | D-cysteine- | 0.34 |
| | polarized light | | | | phenylalanine | |
| L-P⁺²⁰NP | Left circularly | | | | L-cysteine- | 0.35 |
| | polarized light | | | | phenylalanine | |
| D-P⁻²⁵NP | Right circularly | 25 | min | | D-cysteine- | 0.37 |
| | polarized light | | | | phenylalanine | |
| L-P⁺²⁵NP | Left circularly | | | | L-cysteine- | 0.39 |
| | polarized light | | | | phenylalanine | |
| L-P⁻¹⁰NP | Right circularly | 10 | min | | L-cysteine- | 0.1 |
| | polarized light | | | | phenylalanine | |
| L-P⁻²⁰NP | Right circularly | 20 | min | | L-cysteine- | 0.2 |
| | polarized light | | | | phenylalanine | |
| D-P⁺¹⁰NP | Left circularly | 10 | min | | D-cysteine- | 0.1 |
| | polarized light | | | | phenylalanine | |
| D-P⁺²⁰NP | Left circularly | 20 | min | | D-cysteine- | 0.2 |
| | polarized light | | | | phenylalanine | |
| L-P^{0:10}NP | Linearly | 10 | min | | L-cysteine- | 0.13 |
| | polarized light | | | | phenylalanine | |
| L-P^{0:20}NP | Linearly | 20 | min | | L-cysteine- | 0.26 |
| | polarized light | | | | phenylalanine | |
| D-P^{0:10}NP | Linearly | 10 | min | | D-cysteine- | 0.12 |
| | polarized light | | | | phenylalanine | |
| D-P^{0:20}NP | Linearly | 20 | min | | D-cysteine- | 0.24 |
| | polarized light | | | | phenylalanine | |
| D-NP | No | 90 | min | | D-cysteine- | 0.09 |
| | | | | | phenylalanine | |
| L-NP | No | | | | L-cysteine- | 0.09 |
| | | | | | phenylalanine | |
| NP | No | | | *Gold | No chiral | 0 |
| | | | | nanoprism | ligand | |
| NP-D-CYP | No | | | | D-cysteine- | 0 |
| | | | | | phenylalanine | |
| NP-L-CYP | No | | | | L-cysteine- | 0 |
| | | | | | phenylalanine | |
| NS-D-CYP | No | 4 | h | **Gold | D-cysteine- | 0 |
| | | | | nanosphere | phenylalanine | |
| NS-L-CYP | No | 4 | h | | L-cysteine- | 0 |
| | | | | | phenylalanine | |
| L-NP-C | No | 90 | min | Gold | L-cysteine- | 0.03 |
| L-P⁺NP-C | Left circularly | 30 | min | nanocube | phenylalanine | 0.05 |
| | polarized light | | | | | |
| D-NP-C | No | 90 | min | | D-cysteine- | 0.03 |
| D-P⁻NP-C | Right circularly | 30 | min | | phenylalanine | 0.06 |
| | polarized light | | | | | |
| L-NP-O | No | 90 | min | Gold | L-cysteine- | 0.02 |
| L-P⁺NP-O | Left circularly | 30 | min | octahedron | proline | 0.04 |
| | polarized light | | | | | |
| D-NP-O | No | 90 | min | | D-cysteine- | 0.02 |
| D-P⁻NP-O | Right circularly | 30 | min | | proline | 0.04 |
| | polarized light | | | | | |

Note:
*Gold nanoprisms used as seed, growth under reduction Au3⁺ to Au⁺by ascorbic acid, after that, the production modified with chiral ligands.
**Gold nanospheres modified with chiral ligands.

### Experimental Data

Synthesis of Gold L- or D-. Nanoparticles without Illumination

In a series of preliminary experiments gold L-nanoparticles (L-NPs) or D-nanoparticles (D-NPs) were grown from gold nanoprism seeds using a variety of surface ligands without illumination during the growth period to determine which surface ligands provided grown NPs with the best chiroptical signals. Prior to use, nanoprism seeds were centrifuged three times at 6,200g for 5 minutes and re-suspended in 1 mM CTAB at a concentration of 0.5 mM. To synthesize L-NPs or D-NPs, a growth solution was formed by adding 0.8 mL of 10 mM CTAB to 3.95 mL of deionized water. Then 0.2 mL of 10 mM HAuCl₄ was added to form an AuBr₄⁻ complex. After incubation for 5-10 minutes, 0.475 mL of 40 mM ascorbic acid was injected rapidly into the growth solution to reduce Au³⁺ to Au⁺. Then, 5 μL of 4 mM L- amino acid, D- amino acid, L-peptide or D-peptide and 50 μL of 0.5 mM of seeds were injected into the growth solution, mixed thoroughly, and placed at room temperature for 90 minutes. The evolution of the growing L-NPs or D-NPs was monitored by scanning electron microscopy (SEM), circular dichroism, g-factor, zeta potential, and dynamic light scattering (DLS) size analysis, and occurred during a growth period of 90 minutes. The circular dichroism spectra were obtained using aqueous dispersions and revealed distinct chiroptical bands at ˜629 nm and ˜800 nm in the visible/near-infrared window. The morphological development during the growth period was characterized by SEM. Energy-dispersive X-ray spectroscopy (EDX) mapping images for Au, C, N, and O on the NPs were conducted. For the synthesis of L- or D-NP-C, gold nanocubes were centrifuged three times (1,500 ×g, 10 min) and re-suspended in 5 mM CTAC. The growth solution was formed by adding 1 mL of 0.15 M CTAC and 0.2 mL of 10 mM HAuCl₄ to 3.9 mL of deionized water. After incubation for 5-10 minutes, 0.475 mL of 0.15 M ascorbic acid was injected rapidly into the growth solution to reduce Au³⁺ to Au⁺. Then, 5 μL of 1 mM L- or D-CYP and 50 μL of seeds were injected into the growth solution, mixed thoroughly, and placed at room temperature for 90 min. For the synthesis of L- or D-NP-O, gold octahedrons were centrifuged twice (16,200 ×g, 15 min) and dispersed in 5 mM CTAC. The growth solution was formed by adding 1 mL of 0.18 M CTAC and 0.2 mL of 10 mM HAuCl₄ to 3.9 mL of deionized water. After incubation for 5-10 minutes, 0.475 mL of 0.3 M ascorbic acid was injected rapidly into the growth solution. Then, 5 μL of 1.2 mM L- or D-cysteine-proline (L- or D-CPR) and 50 μL of seeds were injected into the growth solution, mixed thoroughly, and placed at room temperature for 90 min.

It was found in these preliminary experiments that only the dipeptides L-cysteine-phenylalanine or D-cysteine-phenylalanine produced L-NPs or D-NPs, respectively, having significant chiroptical signals, with g-factor values ranging from 0.090 to 0.095. Other amino acids or peptides that were tested as surface ligands and found to be ineffective in producing significant chiroptical signals, as defined by the g-factor measured, included: arginine, phenylalanine, glutamic acid, penicillamine, histidine, cysteine, N-isobutyryl-L-cysteine, N-acetyl-L-cysteine, L-cysteine methyl ester hydrochloride, cysteine-arginine, cysteine-glycine, phenylalanine-phenylalanine, cysteine-proline, cysteine-threonine, cysteine-histidine, glutathione, cysteine-phenylalanine-cysteine, phenylalanine-cysteine-phenylalanine, and cysteine-cysteine-phenylalanine. The dipeptide cysteine-tryptophan did produce nanoparticles with some chiroptical signal; however, it was less than half the signal that was produced by the dipeptide cysteine-phenylalanine. No results were seen with a series of tested tetrapeptides or longer peptides. Based on these preliminary experimental results the NPs prepared according to the present disclosure were developed using L-cysteine-phenylalanine or D-cysteine-phenylalanine as the stabilizing surface ligand.

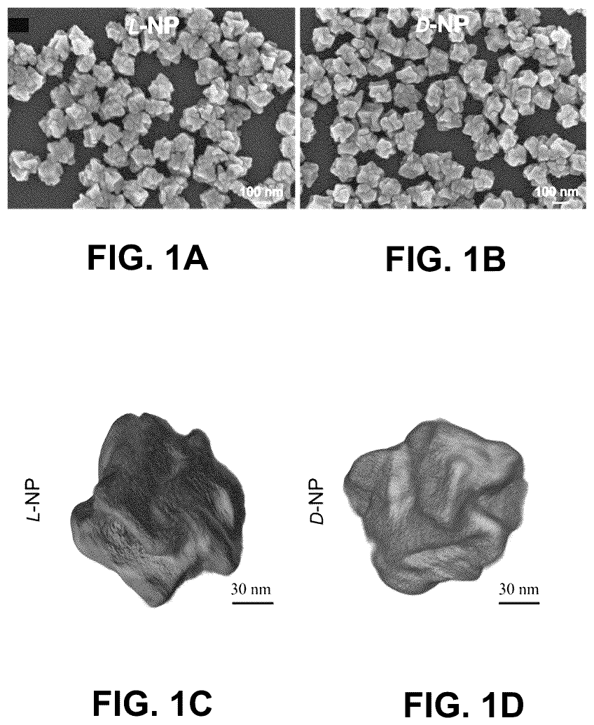

Examples of L-NPs and D-NPs produced without illumination using L-cysteine-phenylalanine or D- cysteine-phenylalanine are shown in FIG. 1A to FIG. 1D. FIG. 1A is a SEM photograph of gold L-NPs prepared using L-cysteine-phenylalanine without any illumination while FIG. 1B is a SEM photograph of gold D-NPs prepared using D-cysteine-phenylalanine without any illumination. The results show fairly homogeneous sizes of L-NPs or D-NPs produced. FIG. 1C is a 3D reconstruction transmission electron microscopy (TEM) tomography image of a gold L-NP prepared using L-cysteine-phenylalanine without any illumination while FIG. 1D is a 3D reconstruction TEM tomography image of a gold D-NP, prepared using D-cysteine-phenylalanine without any illumination. One can see the handedness of the produced L-NPs or D-NPs. The final structures were single crystals with a high-index interface at the edges ((311), (711), (511), and (155)). The EDX-mapping images for Au, C, N, and O on the NPs showed that the dipeptides were evenly distributed over the surface of the NPs.

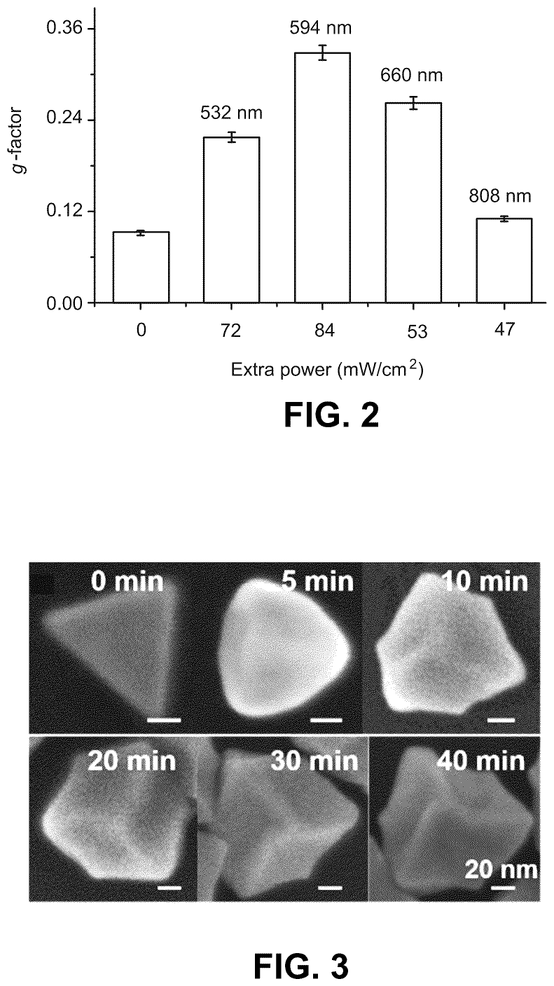

In a next series of experiments NPs were prepared from gold nanoprism seeds using linearly polarized light (LPL) at a series of different wavelengths and light intensities to determine the most effective wavelength and light intensity for growing L-P^{X}NPs. It was found that starting from gold nanoprism seeds or octahedrons the most effective wavelength was 594 nm; for gold nanocubes the most effective wavelength was 532 nm. All wavelengths were most effective at an intensity of 84 mW/cm². Thus, when using gold nanoprisms or octahedrons as the seed the polarized light is set to a wavelength of 594 nm and an intensity of 84 mW/cm² while when using nanocubes as the seed the polarized light is set to a wavelength of 532 nm and an intensity of 84 mW/cm². FIG. 2 shows some of the data generated for growing L-P⁰NPs from nanoprism seeds. As can be seen the greatest increase in g-force is with a light intensity of 84 mW/cm² at a wavelength of 594 nm. Measurement by DLS of the size of the formed L-P^{+X}NPs from gold nanoprism seeds according to the present disclosure showed that the nanoprism seeds had an average edge length of 75±3 nm and the final L-P^{+X}NPs after 30 to 40 minutes of exposure to left circularly polarized light at a wavelength of 594 nm and an intensity of 84 mW/cm² had an average size of approximately 120 nm and they were relatively homogeneous in size distribution.

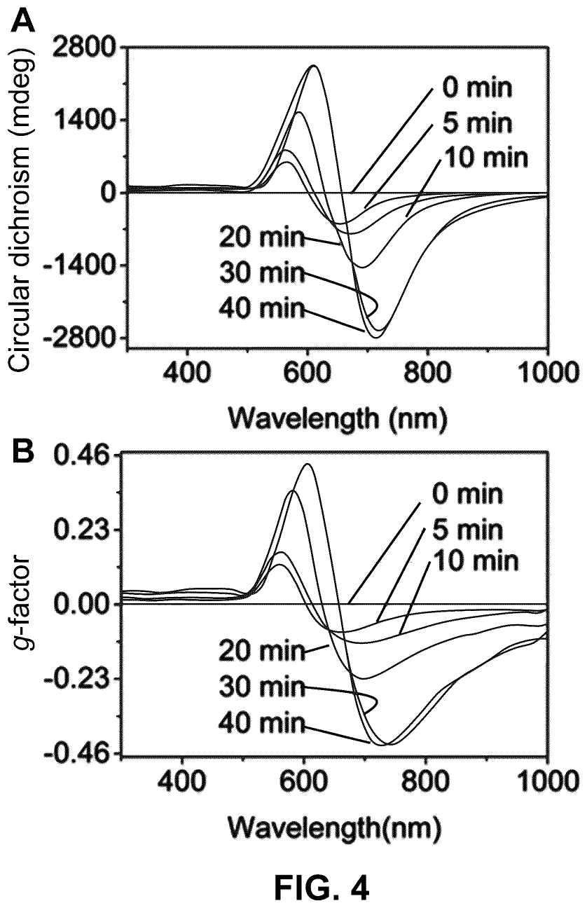

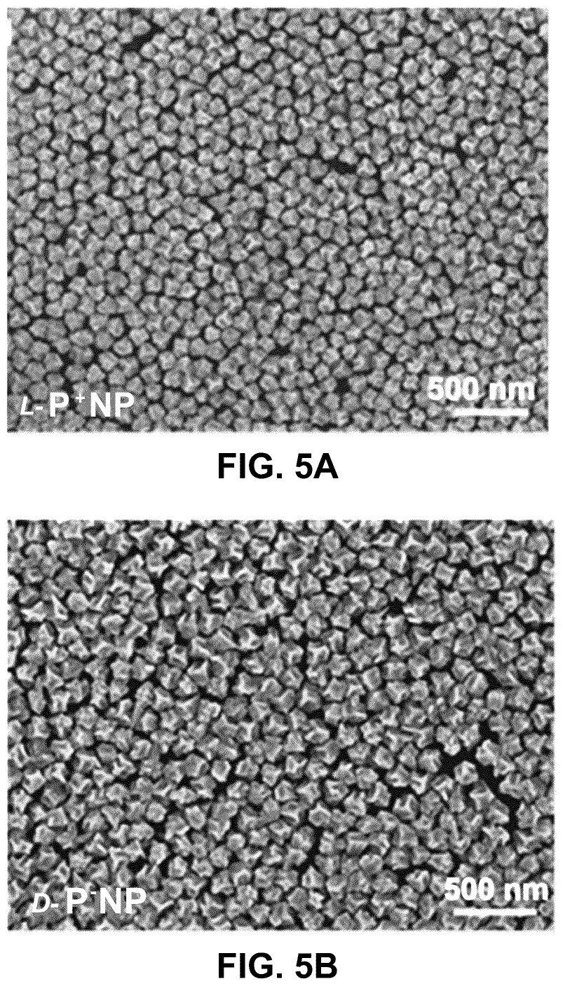

In a next series of experiments the time course of NP growth from gold nanoprism seeds subjected to left or right circularly polarized light at a wavelength of 594 nm and an intensity of 84 mW/cm² in the described growth solution using L or D-cysteine-phenylalanine as the surface ligand. The L/D-P^{X}NPs were grown as described in the methods section of this disclosure. An example of the time course of growth as monitored by SEM images is shown in FIG. 3. Here the growth of L-P⁺NPs, using as surface ligand L-CYP and left circularly polarized light at 594 nm and an intensity of 84 mW/cm²is shown over 0 to 40 minutes. At time 0 one sees the prism shape and over the time course of the growth this shape is dramatically changed. FIGS. 4A and 4B show the circular dichroism spectra and g-factor spectra, respectively, of L-P⁺NPs after 0, 5, 10, 20, 30 and 40 minutes of left circularly polarized illumination at 594 nm at an intensity of 84 mW/cm² according to the present disclosure. One sees that there is little difference in the spectra at 30 minutes versus 40 minutes. This indicates the reaction is completed within 30 minutes under these conditions. Thus, 30 minutes of growth using nanoprisms and the disclosed conditions was selected going forward. FIG. 5A is an SEM image of nanoscale enantiomers of L-P⁺NPs formed after 30 minutes of left circularly illumination at 594 nm at an intensity of 84 mW/cm2 according to the present disclosure while FIG. 5B is an SEM image of nanoscale enantiomers of D-P⁺NPs formed after 30 minutes of right circularly polarized illumination at 594 nm at an intensity of 84 mW/cm2 according to the present disclosure.

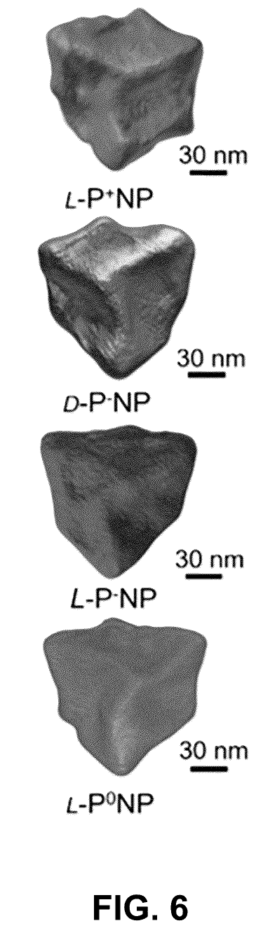

FIG. 6 shows Transmission Electron Microscope (TEM) tomography images of the following nanoparticles prepared according to the present disclosure L-P⁺NP, D-P⁻NP, L-P-NP and L-P⁰NP from nanoprism seeds using polarized illumination of the indicted direction at 594 nm, an intensity of 84 mW/cm² and with the indicated surface The abbreviations of the nanoparticles are shown in Table 1. The L-P⁺NPs were grown in a solution having as the surface ligand L-CYP and used left circularly polarized illumination for 30 minutes. The D-P⁻NPs were grown in a solution having as the surface ligand D-CYP and used right circularly polarized illumination for 30 minutes. The L-P⁻NPs were grown in a solution having as the surface ligand L-CYP and used right circularly polarized illumination for 30 minutes. The L-P⁰NPs were grown in a solution having as the surface ligand L-CYP and used linearly polarized illumination for 30 minutes.

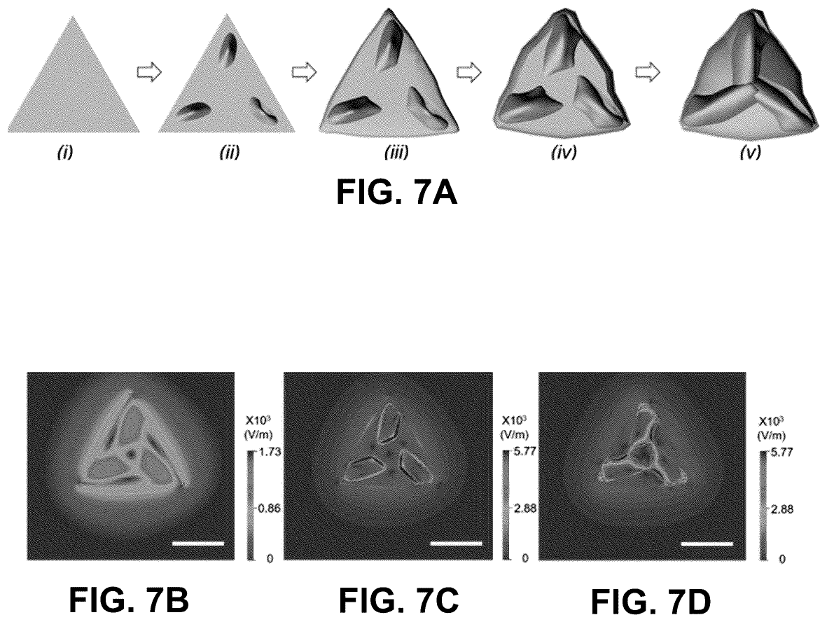

Evolution of morphology, chiroptical activities, and hydrodynamic size of NPs grown under different illumination conditions indicate the light-driven particle growth was accompanied by chirality transfer from photons to NPs resulting in NPs with high-index crystal planes. The NPs obtained after polarized illumination retained some similarity to the original achiral nanoprisms but acquired three out-of-plane protrusions resembling propeller blades that led to strong geometrical and optical asymmetry. This is shown schematically in FIG. 7A for D-P⁻NPs, meaning under right circularly polarized illumination. FIG. 7A shows five, ((i) to (v)), calculated stages of gold deposition based on dynamic formation of “hot spots” on the surface of D-P⁻NPs from nanoprism seeds under illumination by right circularly polarized light. The L-P⁺NPs and D-P⁻NPs exhibited counterclockwise and clockwise rotation of the three blades, respectively. While the handedness of the NPs is determined by their surface ligands, the maximum curvature of the blades, x, is determined by the circular polarization of incident photons. For the same surface ligand, the magnitudes of the x values for L-P⁺NP, L-P⁰NP, and L-P⁻NP (n=5) were 0.029±0.004, 0.023±0.001, and 0.020±0.002, respectively. FIGS. 7B, 7C and 7D show the electric field distributions for stages (i), (iii) and (iv), respectively. The largest effects were always observed at the borders of the NPs for the near field as might be expected based on the typical plasmonic behavior of gold nanostructures.

All of the prepared chiral NPs displayed high colloidal stability and strong chiroptical activity. The NPs with ligands of opposite chirality yielded nearly perfect mirror-symmetrical circular dichroism spectra. For example, the circular dichroism spectra of L-P⁺NPs showed peaks at 605 (+) and 727 (−) nm, while those of D-P⁻NPs displayed peaks in the same positions and with similar intensities, but opposite sign. Circularly polarized illumination results in a considerably enhanced optical asymmetry g-factors that reached values as high as 0.42 at 605 (+) nm and 0.44 at 727 (−) nm for L-P⁺NPs. To the best of our knowledge, these are the highest g-factors obtained for both singular NPs and their assemblies to date. L-P⁻NPs and L-P⁰NPs also exhibited high g-factors of 0.25 and 0.32 at ˜600 (+) nm and 0.22 and 0.3 at ˜700 (−) nm, respectively. The circular dichroism spectrum of D-P⁻NPs was mirror-image symmetrical to that of L-P⁺NPs with peaks at 610 (−) and 732 (+) nm and equally high g-factors of 0.41 and 0.42, respectively. Note that the NPs synthesized without illumination, that is, L-NPs and D-NPs, displayed maximum g-factors of 0.09 at 622 nm, respectively, which were 4.9-fold smaller than those illuminated with CPL. Note also that the g-factors and the curvature of the blades, x, are interdependent because the nanoscale dimensions of the blades result in strong asymmetric interaction with photons.

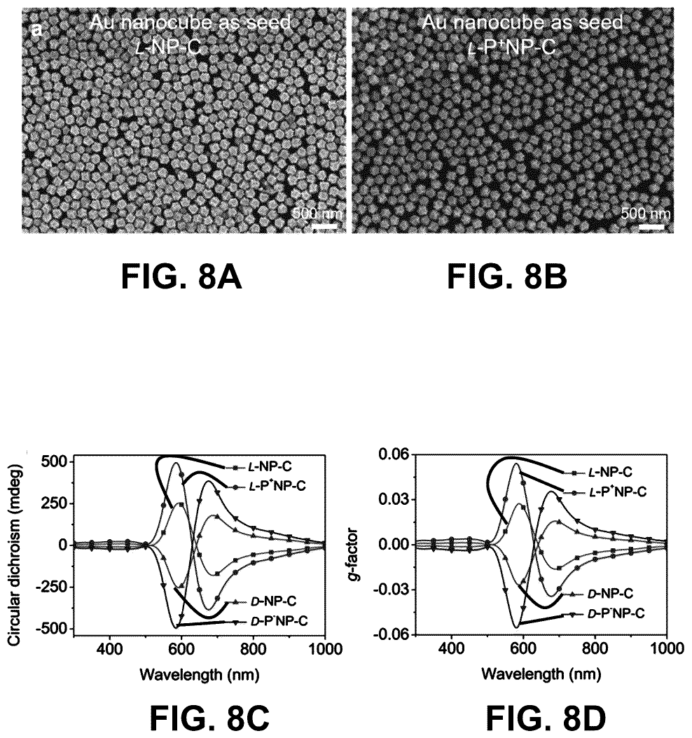

FIG. 8A to FIG. 8D show data obtained using as the seeds gold nanocubes to generate the NPs. FIG. 8A is an SEM photograph of L-NP-C nanoparticles that were grown without illumination, using L-cysteine-phenylalanine as the surface ligand and reaction for 90 minutes as described herein for NP grown in the absence of illumination. FIG. 8B is an SEM photograph of L-P⁺NP-C nanoparticles that were grown with illumination at 532 nm an intensity of 84 mW/cm² using L-CYP as the surface ligand for 30 minutes. FIG. 8C shows the circular dichroism spectra for L-NP-C, L-P⁺NP-C, D-NP-C and D-P⁻NP-C, see Table 1 for the definitions and conditions of growth. FIG. 8D shows the g-factor for L-NP-C, L-P⁺NP-C, D-NP-C and D-P⁻NP-C derived from FIG. 8C. Once can see using circularly polarized light enhanced the g-factor of the grown NPs compared to no illumination by approximately two-fold. The enhanced g-factors using nanocubes were not nearly as high as that seen using gold nanoprisms as the seeds as shown in FIG. 4B.

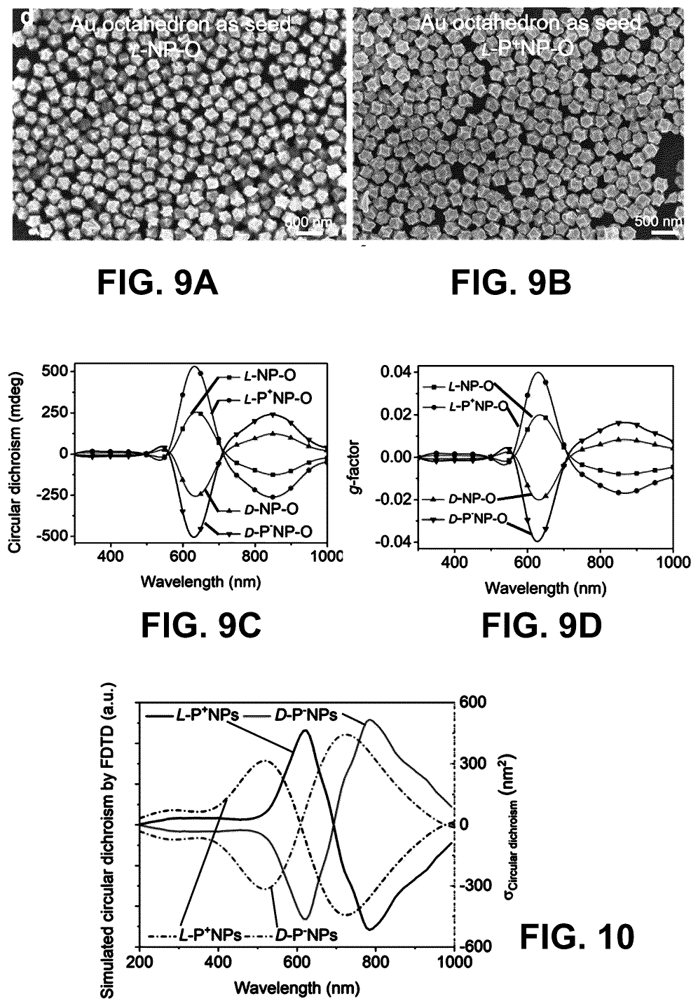

FIG. 9A to FIG. 9D show data obtained using as the seeds gold octahedrons to generate the NPs. FIG. 9A is an SEM photograph of L-NP-O nanoparticles that were grown without illumination, using L-cysteine-phenylalanine as the surface ligand and reaction for 90 minutes as described herein for NP grown in the absence of illumination. FIG. 9B is an SEM photograph of L-P⁺NP-O nanoparticles that were grown with illumination at 532 nm an intensity of 84 mW/cm² using L-CYP as the surface ligand for 30 minutes. FIG. 9C shows the circular dichroism spectra for L-NP-O, L-P⁺NP-O, D-NP-O and D-P⁻NP-O, see Table 1 for the definitions and conditions of growth. FIG. 9D shows the g-factor for L-NP-O, L-P⁺NP-O, D-NP-O and D-P⁻NP-O derived from FIG. 9C. Once can see using circularly polarized light enhanced the g-factor of the grown NPs compared to no illumination by approximately two-fold. The enhanced g-factors using octahedrons as seeds were not nearly as high as that seen using gold nanoprisms as the seeds as shown in FIG. 4B.

To identify the mechanisms underlying the CPL-mediation of chiral NPs and their growth patterns, finite-difference time-domain (FDTD) and semi-empirical density functional theory (DFT) simulations of particle growth were carried out. The observed shapes of the NPs can be explained by regioselective gold deposition on dynamically changing hot-spots and localized reduction of Au(III) to Au(0). Since the electrical field is strongly localized in the corners of the trigonal nanoprisms used as the seeds, the shape of the forming NPs strongly depends on CPL. Using iterative modeling for progressive deposition of gold on gradually changing hot spots, we achieved a model of the final particle geometry with out-of-plane Au segments, which matched the key features of NP geometry observed by TEM tomography, shown in FIG. 6. The simulated circular dichroism spectra for modelled NPs, shown in FIG. 10, are nearly identical to those obtained experimentally for L-P⁺NPs and D-P⁻NPs. The growth mechanism was confirmed for the synthesis of chiral NPs induced by CPL, starting from gold nanocubes and octahedrons using CYP and CPR (cysteine-proline) dipeptides as ligands, which also showed remarkably high chiroptical activity, see FIG. 8A-D and FIG. 9A-D. FIG. 10 shows the simulated circular dichroism by FDTD, solids lines, and the a circular dichroism, dot-dash lines, from the NP model shown in FIG. 7A at stage (v) using Limerical software and differences in extinction coefficients of L-P⁺NPs and D-P⁻NPs models obtained by electrodynamics calculations.

FIG. 11A to 11C show the modeling used to calculate the chirality measures of L-P⁺NPs based on the TEM tomography images of L-P⁺NPs from FIG. 6. Dividing the L-P⁺NPs with the octants of the coordinate system is shown in FIG. 11B-11C. FIG. 12A to 12C show the modeling used to calculate the chirality measures of D-P⁻NPs based on the TEM tomography images of D-P⁻NPs from FIG. 6. Dividing the D-P⁺NPs with the octants of the coordinate system is shown in FIG. 12B-12C.

Hausdorff chirality measure (HCM) and Osipov-Pickup-Dunmur (OPD) index were calculated from three independent calculations using the described method with the point group from the octants. See J-Y. Kim, J Yeom, G. Zhao, H. Calcaterra, J. Munn, P. Zhang, N. A. Kotov, Assembly of Gold Nanoparticles into Chiral Superstructures Driven by Circularly Polarized Light, Journal of American Chemical Society, 2019, 141, 30, 11739-11744. HCM for L-P⁺NPs and D-P⁻NPs are 0.0969±0.0278 and 0.0774±0.0187, respectively, showing similar quantitative degrees of chirality. Moreover, the signs of the OPD indexes are opposite for L-P⁺NPs and D-P⁻NPs, 0.3420±0.1014 and −0.2405±0.0140, respectively. Both of these measures indicate that the synthesized NPs are true geometrical enantiomers. Thus, the circular dichroism amplitude and maximal g-factors can be used as asymmetry measures of the NPs, which is needed to assess the link between nanoscale chirality and immune response.

Distinct nanoscale chirality, high colloidal stability, and biological robustness of chiral gold NPs made them suitable for evaluation of in-vivo and in-vitro immune responses. Both L-P⁺NPs and D-P⁻NPs were stable for at least 12 hours in Dulbecco's phosphate-buffered saline (DPBS) and in the cell culture medium. In addition, neither L-P⁺NPs or D-P⁻NPs caused any change in cellular viability of mouse bone-marrow-derived dendritic cells (BMDCs) or mouse bone-marrow-derived macrophages (BMMs) in culture after 12 hours of exposure and at levels of up to at least 4 nM of the NPs. It was found the PEGylation, as described herein, of the NPs increased their stability and circulation time in vivo. Having established that pure populations of chiral L-NPs and D-NPs can be created with high efficiency the L versus D nanoparticles were tested in a variety of in vitro and in vivo settings to determine their effects. As shown herein, using gold nanoprism seeds, a growth solution having L-cysteine-phenylalanine as the surface ligand, and left circularly polarized light at 594 nm and an intensity of 84 mW/cm² for 30 minutes produced L-P⁺NPs having a g-factor of 0.44. As shown herein, using gold nanoprism seeds, a growth solution having D-cysteine-phenylalanine as the surface ligand, and right circularly polarized light at 594 nm and an intensity of 84 mW/cm² for 30 minutes produced D-P⁻NPs having a g-factor of 0.42. These two populations of nanoparticles were tested in a both in vitro and in vivo settings to determine their effects on immune responses.

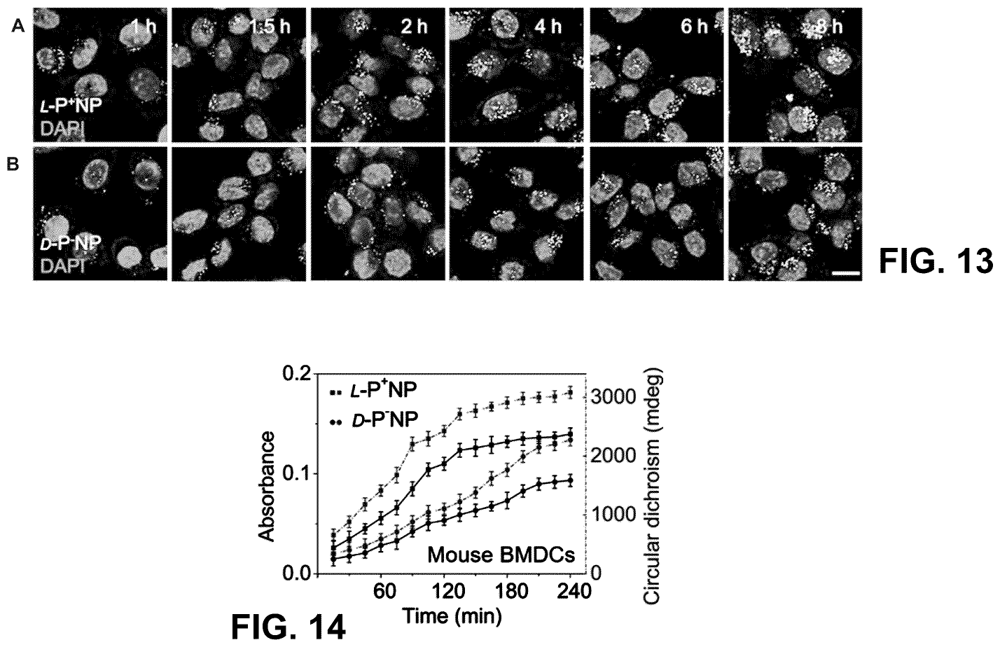

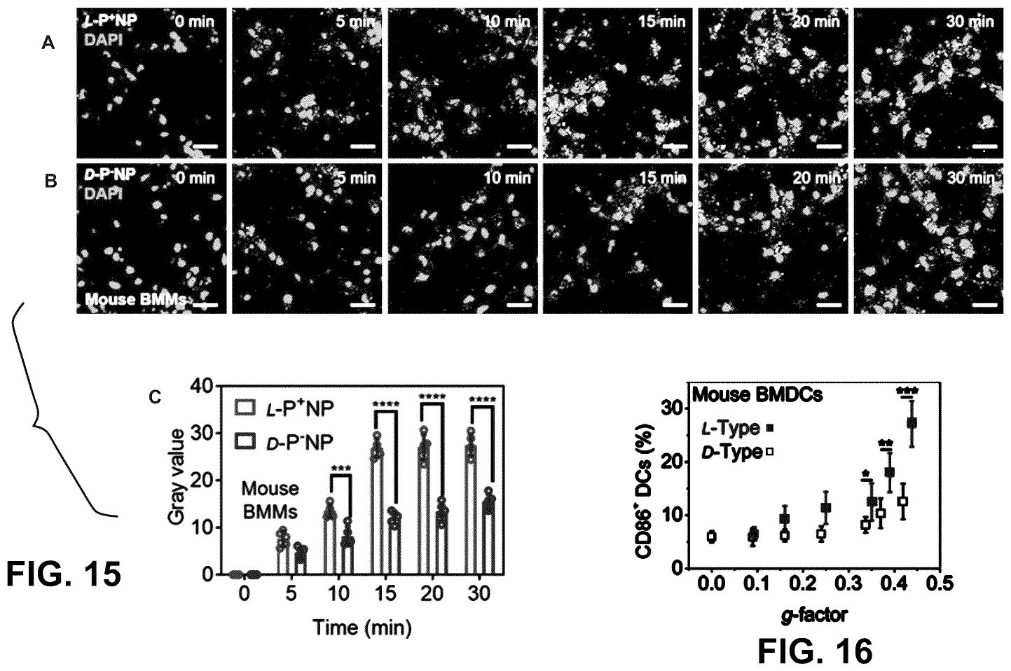

The incubation of mouse BMDCs and BMMs with NP enantiomers showed that L-P⁺NPs exhibited two-fold greater entry efficiency than D-P⁻NPs, as determined by several independent experimental methods: two-photon luminescence (TPL), circular dichroism, and absorbance spectra as shown in FIG. 13, FIG. 14 and FIG. 15. FIG. 13 shows two-photon luminescence staining for 4′,6-diamidino-2-phenylindole (DAPI) and chiral nanoparticles of cultured mouse bone marrow dendritic cells (BMDCs) incubated with either L-P⁺NPs or D-P⁻NPs at 2 nM for the indicated time periods, FIG. 13A are for L-P⁺NPs and FIG. 13B are for L-P⁺NPs. FIG. 14 shows the uptake of L-P⁺NPs (solid squares) or D-P⁻NPs (solid circles) over time as measured by absorbance (solid lines) or circular dichroism (dotted lines) in the cultured mouse bone marrow dendritic cells incubated with either L-P⁺NPs or D-P⁻NPs at 2 nM for the indicated time periods. The data shows that the L-P⁺NPs were taken up at a rate and to an extent that was two-fold greater than that of the D-P⁻NPs. Similar results were found with incubation of either L-P⁺NPs or D-P⁻NPs at 2 nM with mouse bone-marrow-derived macrophages (BMMs) as shown in FIGS. 15A, 15B and 15C. Here also the L-P⁺NPs were taken up to a greater extent and faster than D-P⁻NPs. FIGS. 15A and B show two-photon luminescence staining for 4′,6-diamidino-2-phenylindole (DAPI) and chiral nanoparticles of cultured mouse bone marrow dendritic cells incubated with either L-P⁺NPs (A) or D-P⁻NPs (B) at 2 nM for the indicated time periods, scale bars are 20 micrometers, FIG. 15 C shows quantification of the gray value of the white dots from the L-P⁺NPs (A) or D-P⁻NPs at each time point with the first bar of each time point in 15C being the L-P⁺NPs and the second bar being the D-P⁻NPs, the *** is significance at P<0.001 and **** is significance at P<0.0001 calculated by one-way ANOVA and data is presented as mean±s.d. with n=5. To eliminate the possibility that the difference in entry efficiency was associated with chirality of the surface ligand peptides (CYP) rather than the chirality of the NPs, the CYP dipeptide stabilizers were removed from the surface of the NPs by incubation with dithiothreitol (DTT) and the experiments were repeated. It was found that the cellular uptake depended only on the chiral configuration of the NP itself and was not affected by the chirality of surface ligand CYP. Removal of the CYP was confirmed by multiple reaction monitoring liquid chomotography mass spectrometry. In addition, testing of g-factor before and after removal of the surface ligands showed that the chirality was retained confirming that the chiral signal of NPs comes from the nanoscale configuration rather than the chiral ligands on the surface. The data confirms that L-P⁺NPs are more effectively taken up in both BMDCs and BMMs compared to D-P⁻NPs and that it is due to the nanoscale chirality of the NPs rather than the chirality of the surface ligands.

Using L-P^{+X}NPs and D-P^{-X}NPs of varied g-factor values, created by different times of exposure to illumination, one can expose cells to NPs having varied chirality. The results of exposing mouse BMDCs to L-type NPs (solid squares) or D-type NPs (open squares) with varied g-factor values for 12 hours at 2 nM are shown in FIG. 16. The mouse BMDCs were cultured with 2 μg/mL monophosphoryl lipid A (MPL), 20 μg/mL OVA and nanoprisms (g-factor 0.0), L-NPs (g-factor 0.09), D-NPs (g-factor 0.09), L-P⁺¹⁰NPs (g-factor 0.16), D-P⁻¹⁰NPs (g-factor 0.16), L-P⁺¹⁵NPs (g-factor 0.25), D-P⁻¹⁵NPs (g-factor 0.24), L-P⁺²⁰NPs (g-factor 0.35), D-P⁻²⁰NPs (g-factor 0.34), L-P⁺²⁵NPs (g-factor 0.39), D-P⁻²⁵NPs (g-factor 0.37), L-P⁺NPs (g-factor 0.44), and D-P⁻NPs (g-factor 0.42) all NPs at 2 nM for 12 hours and then CD86 plus cells were measured by flow cytometry. In all cases the values for L-type NPs were greater than D-type NPs and the results were significant at g-factors of 0.35 or greater. The data shown are mean±s.d., n=5, and * P<0.005, ** P<0.01,*** P<0.001 analyzed by Student's t-test. The results show that an increase in chirality, as measured by g-factor, increases the activation of the dendritic cells as measured by CD86 plus cell number. In addition, the data shows that the L-type NPs always result in a greater increase that is significant at g-factors of 0.35 and greater.

Lysosomal escape efficiency of L-P⁺NPs was also higher than for D-P⁻NPs, data not shown. Bio-TEM images showed the non-agglomerated states of NPs in different stages of endocytosis, indicating that the geometry of individual particles rather than particle aggregates, determines the uptake and lysosomal escape efficiency of NPs. Electron microscopy data suggest that endocytosis of L-P⁺NPs is faster than that of D-P⁻NPs and involves stronger association with cellular membranes. The monotonic dependence of NP uptake by mouse BMDCs and mouse BMMs on the g-factor of the NPs confirms the dependence of the endocytosis rate on nanoscale chirality of the particles.

Cell culture experiments also showed that the expression of the co-stimulatory biochemical markers CD40, CD80, CD86, SIINFEKL-MHC I, MHC II, pro-inflammatory cytokines interleukin IL-12, and tumor necrosis factor-α (TNF-α) induced by L-P⁺NPs were higher than those by D-P⁻NPs after incubation with mouse BMDCs. Similarly, for mouse BMM cell cultures, interleukin 1 beta (IL-1β), IL-12, and TNF-α, after incubation with L-P⁺NPs, were 1.9-fold (P<0.001), 2.3-fold (P<0.001), and 2.3-fold (P<0.001) higher, respectively, than those for D-P⁻NPs. A distinct correlation between g-factors of the NPs and the immunological response of mouse BMDCs for both left- and right-handed enantiomers, namely L-P⁺NPs and D-P⁻NPs, was observed, whereas the levels of CD86 observed for achiral/racemic nanoprisms and NPs were low as shown in FIG. 16. Concurrently, the level of SIINFEKL-MHC I complexes of mouse BMDCs was 2.1-fold (P<0.001) greater after incubation with L-P⁺NPs compared to D-P⁻NPs. The antigen, ovalbumin, uptake was not affected by any of the NP enantiomers.

Control experiments conducted in conjunction with these findings showed the following: (1) the changes in expression of CD86 remained unchanged after DTT-modification, which removes the surface ligands, of the NPs; (2) activation of immune cells by the dipeptide CYP alone was not observed; and (3) immune responses to symmetric gold prisms, achiral NPs coated with L/D-CYP (120±6 nm), or nanospheres coated with L/D-CYP (30±2 nm) were negligible as were their circular dichroism amplitudes. Thus, one can firmly conclude that (1) the biological response of immune cells to left- and right-handed nanoscale enantiomers is distinctly asymmetric and (2) this difference originates from a sequence of particle-specific biochemical signaling events related to their intracellular uptake.

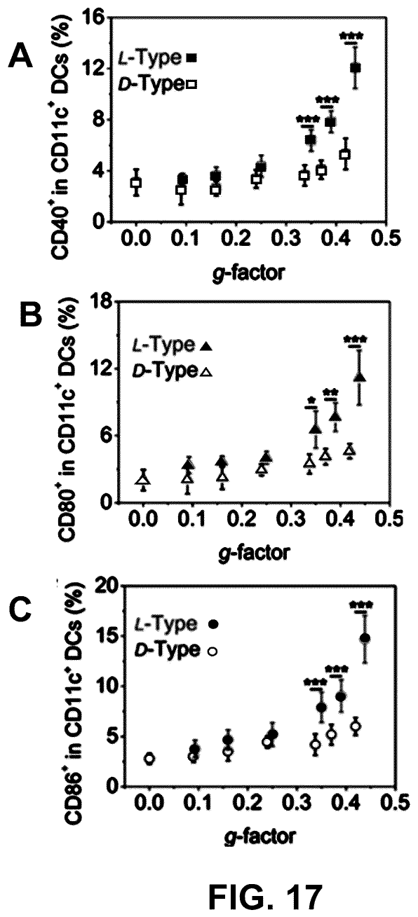

To evaluate whether the immune response asymmetry would be evident in vivo, C57BL/6 female mice, n=5 for each condition, were subcutaneously immunized by 2 mg of LID NPs with different chirality, MPL (10 μg) and OVA (50 μg). Then 36 hours after injection the draining lymph nodes (dLNs) were collected and the expression of CD40, CD80, and CD86 were analyzed by flow cytometry. Flow cytometry showed that the levels of CD40, CD80, and CD86 in recruited dendritic cells, identified by the CD11c⁺ marker, from the dLNs were markedly upregulated 36 hours after stimulation with chiral L/D NPs as shown in FIGS. 17A, 17B and 17C. In the FIG. 17A-17C the solid symbols are the L-P^{+X}NPs and the open symbols are the D-P^{-X}NPs, with g-factor 0.0 being nanoprism seeds. This data is consistent with enhanced mouse BMDCs maturation in-vitro as shown above. The expression levels of CD40, CD80, and CD86, following treatment with L-P⁺NPs with a chirality of 0.44 were 12.06±1.61%, 11.19±2.44%, and 14.68±2.36%, which were 2.27-fold (P<0.001), 2.42-fold (P<0.001), and 2.45-fold (P<0.001), respectively, higher than those of the D-P⁻NPs with a chirality of 0.42, which were 5.32±1.21%, 4.63±0.66%, and 6.00±0.87%, respectively. Similar to the in-vitro response, the expression levels of the cytokines increased as the g-factors of both types of NPs increased. The achiral and racemic particles showed limited enhancement, substantiating the relationship between nanoscale chirality and enhanced immune response.

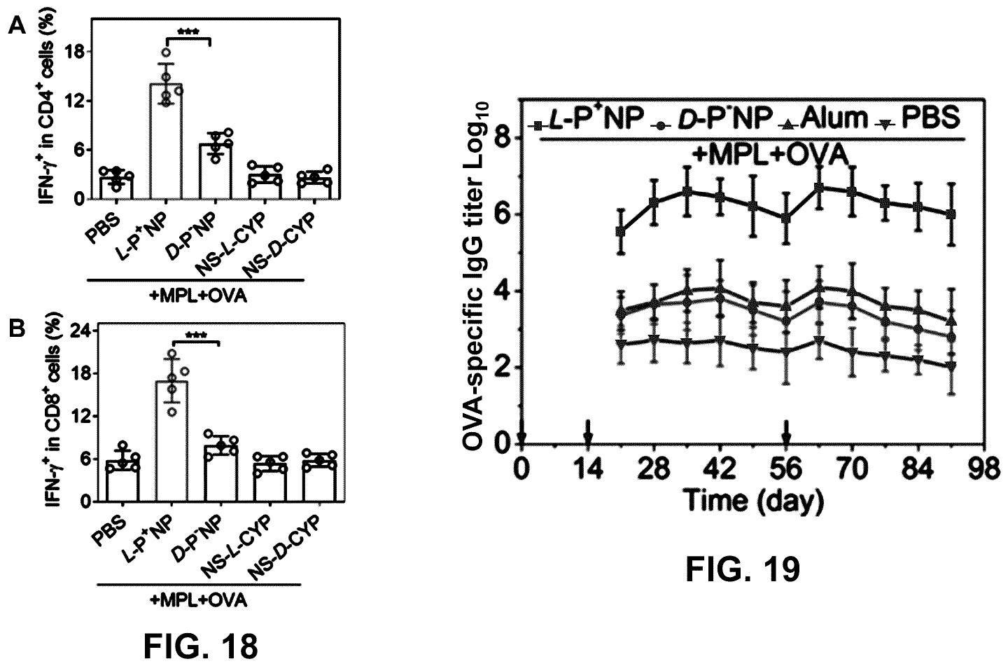

In an additional in vivo testing protocol, C57BL/6 mice (n=5 per group) were subcutaneously immunized with MPL (10 μg)+OVA (50 μg), and then either PBS, 2 mg of NS-L-CYP, 2 mg of NS-D-CYP, 2 mg of L-P⁺NP or 2 mg of D-P⁻NP. At 7 days after immunization the expression of IL-2, IL-4, IL-6, IL-10, and IL-12 in serum were measured by ELISA 7. The expression of all the interleukins measured increased and were the greatest after immunization with L-P⁺NPs, indicating that the left-handed chiral NPs enhanced immune responses in mice better than of D-P⁻NPs. Further evidence of this effect was found from evaluation of the secretion of interferon-γ (IFN-γ) by CD4⁺ and CD8⁺ cells at the same time point. As with interleukins, mice immunized with L-P⁺NPs led to stronger CD4⁺ (2.08-fold) and CD8⁺ (2.15-fold) spleen T cell secretions of IFN-γ than for D-P⁻NPs as shown in FIGS. 18A and 18B, respectively. Although the data is not shown similar results were seen for secretion of tumor necrosis factor-a (TNF-α) by CD4⁺ and CD8⁺ spleen T cells.

The chiral NPs also increased serum antibody titer to OVA with the results from L-P⁺NPs being extremely large compared to D-P⁻NPs. C57BL/6 mice (n=5 per group) were immunized with OVA (50 μg) and MPL plus one of the indicated adjuvants: 50 μL of Alum+10 μg of MPL, 2 mg of D-P⁻NP+10 μg MPL, 2 mg of L-P⁺NP+10 μg of MPL at three time points over 91 days. The serum was collected at the indicated times and the OVA-specific IgG levels were determined. The results are shown in FIG. 19, note the Y-axis is a logarithmic scale Log₁₀. Black arrows indicate the times of immunization, day 0, day 14 and day 56. Data are presented as mean s.d. (n=5). *P<0.05, **P<0.01, ***P<0.001, analyzed by Student's t-test.

The concomitant production of ovalbumin (OVA)-specific antibody after injection of L-P⁺NPs was 1584-fold higher than the production after D-P⁻NPs injection as shown in FIG. 19.

All these data separately and in aggregate demonstrate that the L-P⁺NPs stimulate a stronger in-vivo immune response than the D-P⁻NPs, which matches the findings of the in-vitro experiments. Also, the population of central memory T cells (CD44+ and CD62L*) and effector memory T cells (CD44⁺ and CD62L⁻) within the CD4⁺ and CD8⁺ T cells, after chiral NPs stimulation, were maintained after injection of L-P⁺NPs, data not shown. No obvious histological cytotoxicity was evident in the kidney or liver tissues examined with hematoxylin and eosin (H&E) staining from each group out to 15 days after immunization, data not shown. In addition, measurements of levels of alanine transaminase, aspartate transaminase, blood urea nitrogen (BUN) or creatinine showed no significant changes out to 15 days after injection of mice with either L-P⁺NPs or D-P⁻NPs, data not shown.

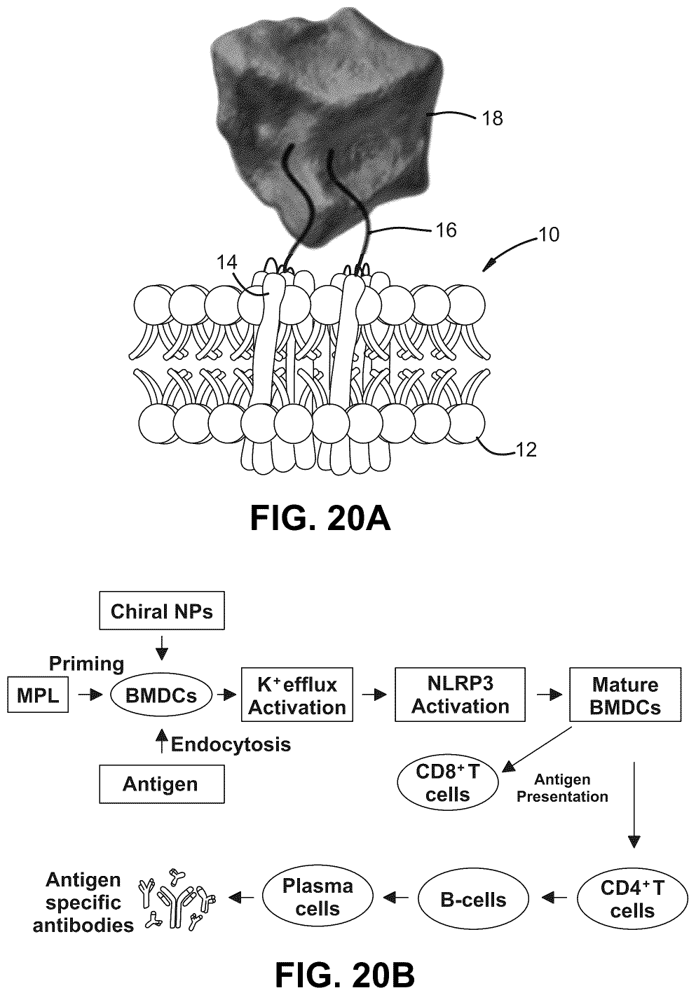

The present inventors explored the biological mechanisms underlying the different immunological response to left- and right-handed NP enantiomers in mouse BMDCs. The large adhesion G protein-coupled receptor family (AGPCR of receptors attracted their attention in particular because they have large flexible extracellular domains, which are easily accessible to NPs. Furthermore, these domains and AGPCR receptors in general are related to adhesion, cell signaling, and cellular uptake. These receptors are also commonly found in many immune cells. The inventors initially studied interactions of epidermal growth factor-like module receptor 1 (EMR1) a receptor typical to mice, then extended the study to cluster of differentiation 97 (CD97), a receptor that is common to both murine and human immune cells. Both of the receptors have an extracellular segment of up to 5-6 epidermal growth factor (EGF) like domains connected into a flexible chain. FIG. 20A shows a schematic of a possible interaction between a chiral NP according to the present disclosure and the receptors while FIG. 20B shows a schematic of the possible in vivo mechanism of induction of immune responses by chiral NPs according to the present disclosure. In FIG. 20A the phospholipid cellular membrane is shown at 10 and the intracellular side is shown at 12. A schematic of an EMR1 receptor is shown at 18 spanning the membrane 10. The receptor 14 includes a plurality of extracellular epidermal growth factor like domains 16, one or more of which interact with a chiral nanoparticle 18 according to the present disclosure.

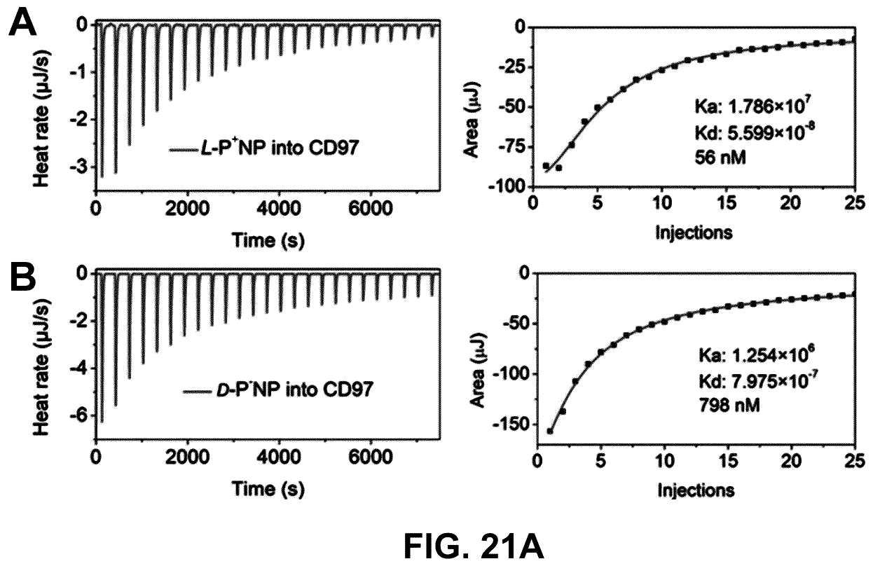

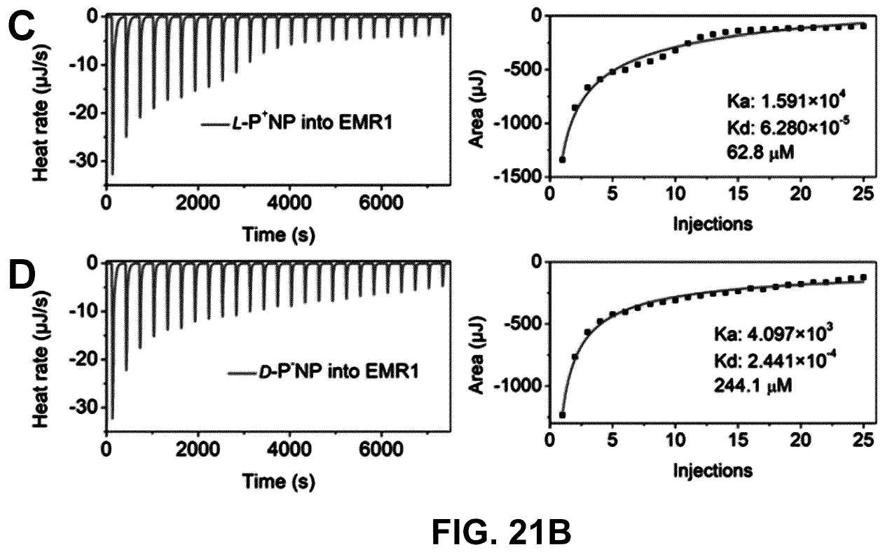

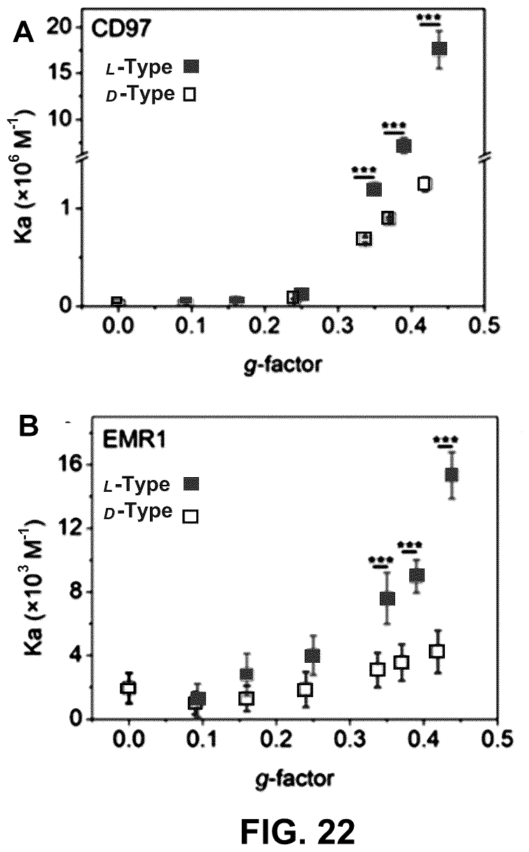

To examine the binding of L-P⁺NPs and D-P⁻NPs to EMR1 and CD97 binding studies were carried out in cell-free buffer solution and using isothermal titration calorimetry (ITC) data. In FIG. 21A to 21D one sees the ITC data for L-P⁺NP (100 μM) and for D-P⁻NP (100 μM) after incubation with CD97 (10 μM) and EMR1 (10 μM) for 30 minutes. FIG. 21A shows data for L-P⁺NP binding to CD97, FIG. 21B shows data for D-P⁻NP binding to CD97, FIG. 21C shows data for L-P⁺NP binding to EMR1 and FIG. 21D shows data for D-P⁻NP binding to EMR1. The process for the ITC procedure is provided above. In FIGS. 22A and 22B the calculated binding affinity (Ka) data is presented for a series of L-P^{+X}NPs and D-P^{-X}NPs having different g-factor values binding to CD97 and EMR1, respectively. Note the Y-axis for binding to CD97 is split to allow for accommodation of the high binding affinity of L-P^{+X}NPs to fit on the graph, solid symbols are L-P^{+X}NPs and open symbols are D-P^{-X}NPs.

The binding affinity, Ka, between L-P⁺NPs and CD97 and EMR1 in cell-free buffer was 14.0+0.9-fold and 3.6+1.2-fold higher than that for D-P⁻NPs with CD97 and EMR1, respectively, as shown in FIG. 22A and FIG. 22B. The absolute values of Kₐ for binding of L-P⁺NPs, these have the highest g-factor, to CD97 and EMR1 are (1.8±0.2)×10⁷ M⁻¹ and (1.5±0.15)×10⁴ M⁻¹, respectively, which is sufficient for assessment of Kₐ for cell signaling events. The absolute values of Kₐ for binding of D-P⁺NPs, these have the highest g-factor, to CD97 and EMR1 are (1.3±0.1)×10⁶ M⁻¹ and (4.2±1.3)×10³ M⁻¹, respectively, which is sufficient for assessment of Kₐ for cell signaling events. The data in FIG. 22A and FIG. 22B also establish that the chirality of the NPs determines its binding affinity as would be expected; as the chirality increased the binding affinity also increased. These values are comparable to typical Kₐ values for receptors, being in the range of 10³ and 10⁹ M⁻¹.

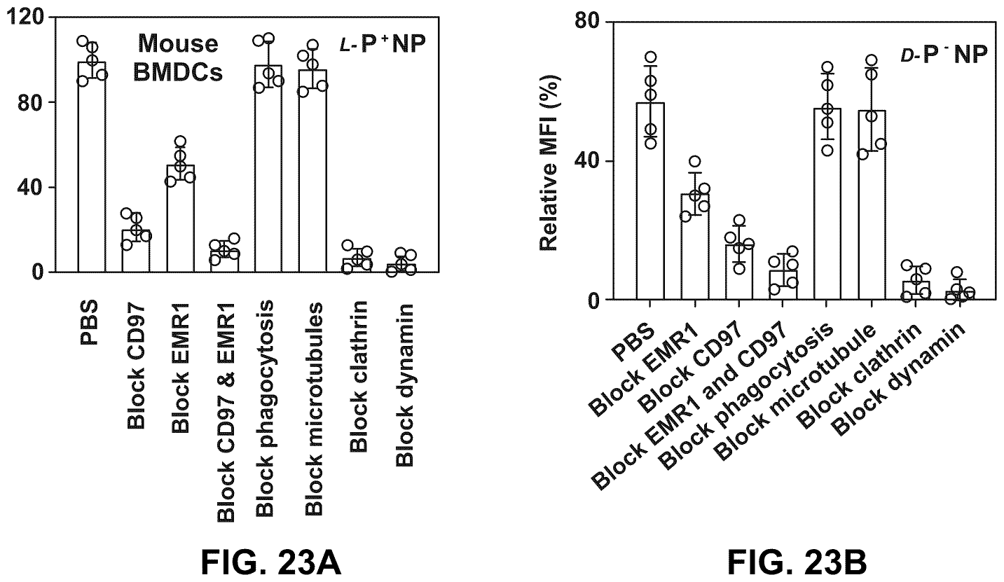

To specifically test whether CD97 and EMR1 are involved in uptake of L-P⁺NPs and D-P⁻NPs by mouse immune cells as suggested in the schematic shown in FIG. 20B, the receptors were blocked by corresponding antibodies and uptake of the NPs was measured by flow cytometry. In additional experiments phagocytosis, microtubules, clathrin or dynamin were blocked by the inhibitors cytochalasin D, nocodazole, chlorpromazine or dynasore, respectively, to determine which were involved in uptake of the NPs. The uptake was monitored by flow cytometry and the results are presented as relative mean fluorescence intensity (MFI) % as is known in the art. FIG. 23A shows the results for L-P⁺NPs and FIG. 23B shows the results for D-P⁻NPs. The mouse BMDCs were pre-treated with PBS, anti-EMR1 (5 μg/mL) antibody, anti-CD97 antibody (10 μg/mL), both anti-EMR1 antibody (5 μg/mL) and anti-CD97 antibody (10 μg/mL), cytochalasin D (phagocytosis inhibitor, 10 μg/mL), nocodazole (microtubule inhibitor, 10 μg/mL), chlorpromazine (clathrin inhibitor, 10 μg/mL) and dynasore (dynamin inhibitor, 10 μg/mL) for 2 hours. Cells were subsequently incubated with L-P⁺NPs or D-P⁻NPs (2 nM) for 12 hours and uptake was measured by flow cytometry.

The endocytosis of both L-P⁺NPs and D-P⁻NPs was reduced to a greater extent by blocking of CD97 than EMR1 because of the higher affinity of CD97 for the NPs. When the two antibodies were used simultaneously, the cellular uptake of both NPs was almost completely inhibited, which indicated that the activity of chiral NPs was mediated by their interactions with CD97 and EMR1 receptors. The data also shows that blocking clathrin or dynamin dramatically reduced uptake of both L-P⁺NPs and D-P⁻NPs, thereby confirming that both L-P⁺NPs and D-P⁻NPs entered the cell through binding to the CD97 and EMR1 with downstream recruitment of dynamin and clathrin in mouse BMDC. The higher binding affinity of L-P⁺NPs to both CD97 and EMR1 causes the higher cellular uptake of L-P⁺NPs versus the D-P⁻NPs.

These findings were confirmed using fluorescence resonance energy transfer (FRET) microscopy during NP uptake between Cy3-labeled L-P⁺NPs and Cy5-labeled receptors. Excitation at 540 nm in the absorption band of Cy3, where the Cy5 label served as a FRET acceptor in this pair, emitted only when NPs and CD97 or EMR1 formed a complex. The localization of L-P⁺NPs with respect to other parts of the cell was monitored by Cy3 emission. After incubation with mouse BMDC, the intensity of red emission from Cy5-CD97 in the cell membrane gradually increased, which indicated L-P⁺NP bound to the extracellular domain of the receptor. As incubation time increased, FRET emission from CD97 further increased, confirming the formation of NP-CD97 complexes. Subsequently, the NPs entered into the cell and the Cy5 intensity on the cytomembrane gradually decreased, while the intracellular Cy3 signal gradually increased indicating endocytosis. For D-P⁻NPs, FRET emission was much weaker but followed a similar pattern. Similar processes were observed for Cy5-EMR1, indicating that both NP-CD97 and NP-EMR1 complexes form, which mediated endocytosis of NPS into mouse BMDC. Two-photon luminescence (TPL) imaging also showed that L-P⁺NPs and D-P⁻NPs became subsequently co-localized with dynamin and clathrin, proving that these two proteins facilitate endocytosis of NPs into mouse BMDCs.

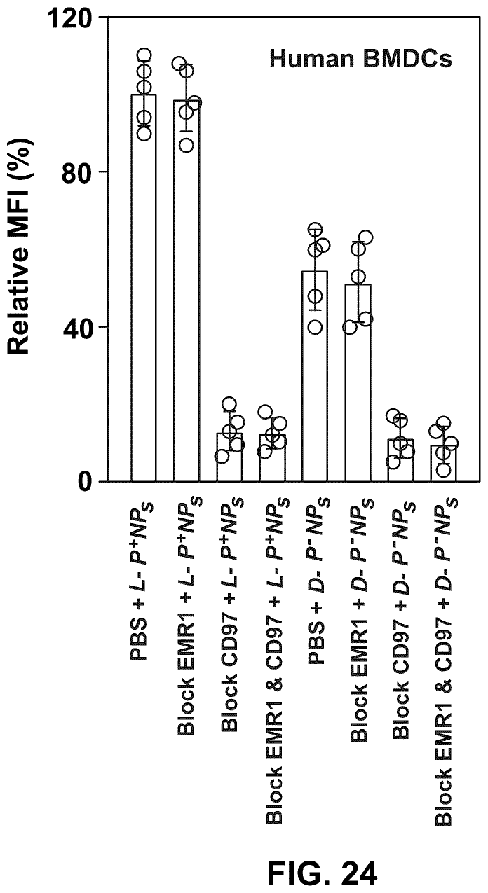

In a next series of experiments the uptake of L-P⁺NPs and D-P⁻NPs by human BMDC, which carry CD97 receptors, was measured. Flow cytometry data for human BMDC after being treated with PBS, anti-EMR1 antibody (30 μg/mL), anti-CD97 antibody (20 μg/mL), both anti-EMR1 antibody (30 μg/mL) and anti-CD97 antibody (20 μg/mL) and then incubated with L-P⁺NP (2 nM) or D-P⁻NP (2 nM) for 8 hours is shown in FIG. 24. As expected, the blockage of CD97 receptors, found on these human cells, dramatically reduced uptake of L-P⁺NPs and D-P⁻NPs. The results with blockage of EMR1 suggest that these receptors are either not found on these cells or do not participate in the endocytosis of NPs unlike in murine cells. The FRET and TPL data reproduced the uptake and localization patterns observed for murine cells, indicating the commonality of the NP endocytosis mediated by AGPCR receptors. The intensity of CD97-Cy5 with L-P⁺NPs was also much higher than with D-P⁻NP as expected.

Concerning the downstream immune response processes as shown in FIG. 20B, the NLR family pyrin domain containing protein 3 (NLRP3) and caspase-1 expression and activation in mouse BMDCs by L-P⁺NPs under a variety of conditions was investigated by confocal imaging. Confocal imaging of NLRP3 inflammasome activation in mouse BMDCs after incubation with PBS, MPL (2 g/mL)+OVA (20 μg/mL), L-P⁺NP+MPL+OVA, L-P⁺NP+MPL+OVA+MCC950 (NLRP3 inhibitor), L-P⁺NP+MPL+OVA+amiodarone (K⁺ channel inhibitor), L-P⁺NP+MPL+OVA+KCl (K⁺ efflux inhibitor), L-P⁺NP+MPL+OVA+dynasore (dynamin inhibitor), L-P⁺NP+MPL+OVA+chlorpromazine (clathrin inhibitor), L-P⁺NP+MPL+OVA+CA-074-Me (cathepsin B inhibitor), L-P⁺NP+MPL+OVA+cytochalasin D (phagocytosis inhibitor), L-P⁺NP+MPL+OVA+N-acetyl-L-cysteine ((NAC) reactive oxygen species (ROS) inhibitor) and L-P⁺NP+MPL+OVA+nocodazole (microtubule inhibitor) for 12 hours, respectively. All NPs were added at a level of 2 nM. In the inhibition studies the cells were pre-treated with MCC950 (10 μM), chlorpromazine (50 μM), cytochalasin D (2 μM), NAC (5 mM), amiodarone (40 μM), KCl (130 mM), dynasore (80 μM), nocodazole (10 μM), or CA-074-Me (5 μM) for 2 hours and then co-cultured (2 nM) or D-P⁺NPs (2 nM) for 12 hours. In the original figures Blue=DAPI, Red=Caspase-1, Green=NLRP3. Scale bar, 20 μm.

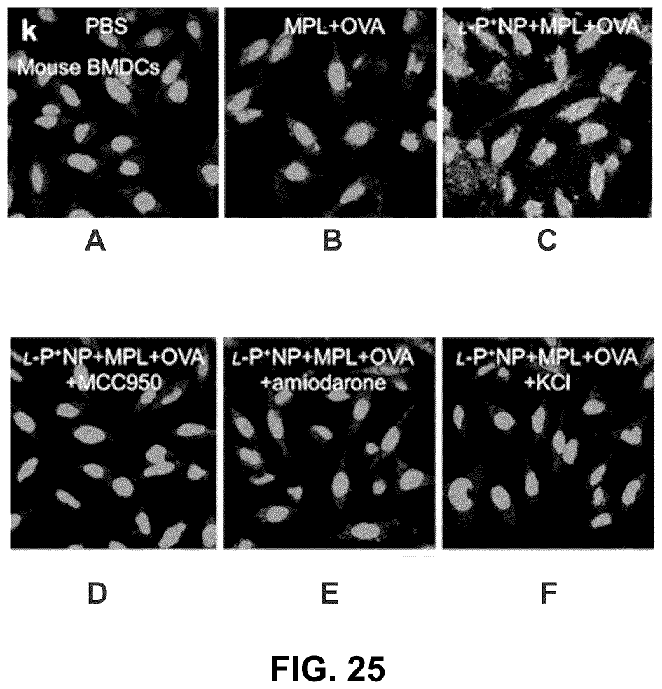

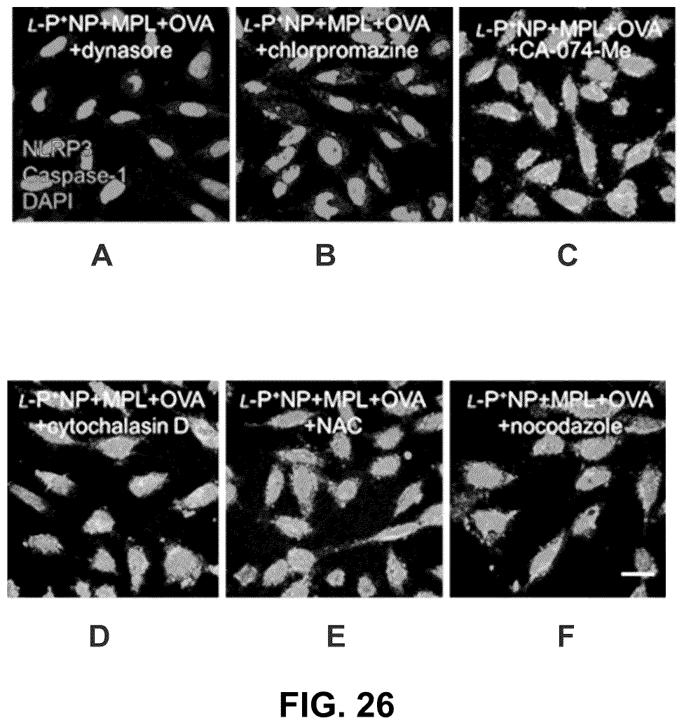

The results for the L-P⁺NPs are shown in FIG. 25A-25F and FIG. 26A-26F. The confocal imaging in FIG. 25C showed that the NLR family pyrin domain containing protein 3 (NLRP3) and caspase-1 were expressed after incubation of mouse BMDCs with L-P⁺NPs for 12 hours, especially the NLRP3. FIG. 25A showed no activation of either NLRP3 or caspase-1 as expected. With MPL and OVA, FIG. 25B, there was a slight activation of NLPP3. The activation by L-P⁺NPs was absent in the presence of MCC950 (25D), KCl (25F) and almost completely absent in the presence of amiodarone (25E). Likewise, the activation was absent in the presence of dynasore (26A) and chlorpromazine (26B), but unaffected by CA-074-Me (26C), cytochalasin D (26D), NAC (26E) and nocodazole 26F). Equally importantly, the L-P⁺NPs displayed stronger inflammasome activation than D-P-NPs. When the K⁺ ion channel was blocked by amiodarone the expression of inflammasomes was significantly attenuated. Incubation of BMDCs with 130 mM KCl, which completely inhibited K⁺ efflux, also demonstrated the central role of the K⁺ ion channel in NP activation of the inflammasome pathway. Additional support for this mechanism is provided by the literature data on K⁺ efflux-mediated inflammasome activation⁴⁴ as well as by ELISA, western blot, and RT-PCR data discussed and shown in this disclosure.

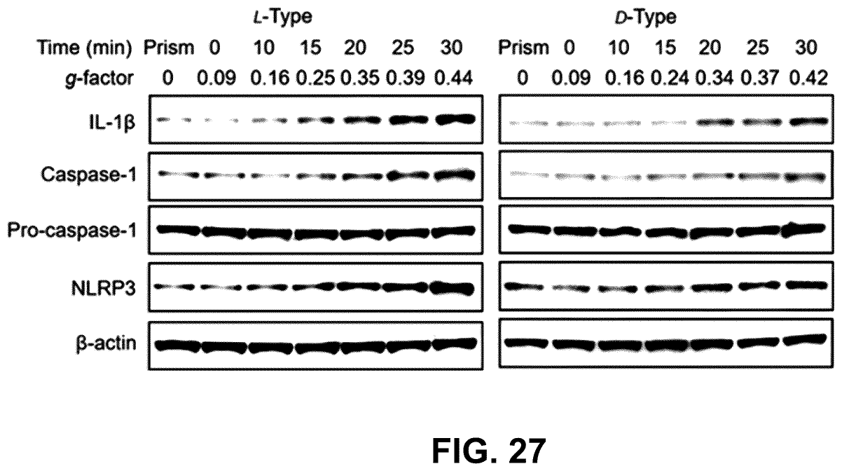

The large difference in immune response for L-P⁺NPs versus D-P⁻NPs enantiomers was further confirmed by Western blot and reverse transcription polymerase chain reaction (RT-PCR) data. As shown in FIG. 27, the Western blot data corroborated the microscopy data, establishing that the expression of NLRP3, IL-1β, and caspase-1 in BMDCs was considerably higher for L-P⁺NPs than for D-P⁻NPs and that expression increased as the g-factor of the NPs increased as shown. The data was generated from mouse BMDCs after incubation with the indicated NPs at 2 nM plus 2 μg/mL MPL and 20 μg/mL OVA. As a negative benchmark experiment, the inflammasomes from BMDC collected from NLRP3 knockout mice (NLRP3^{-/-}) could not be activated by any chiral NPs.

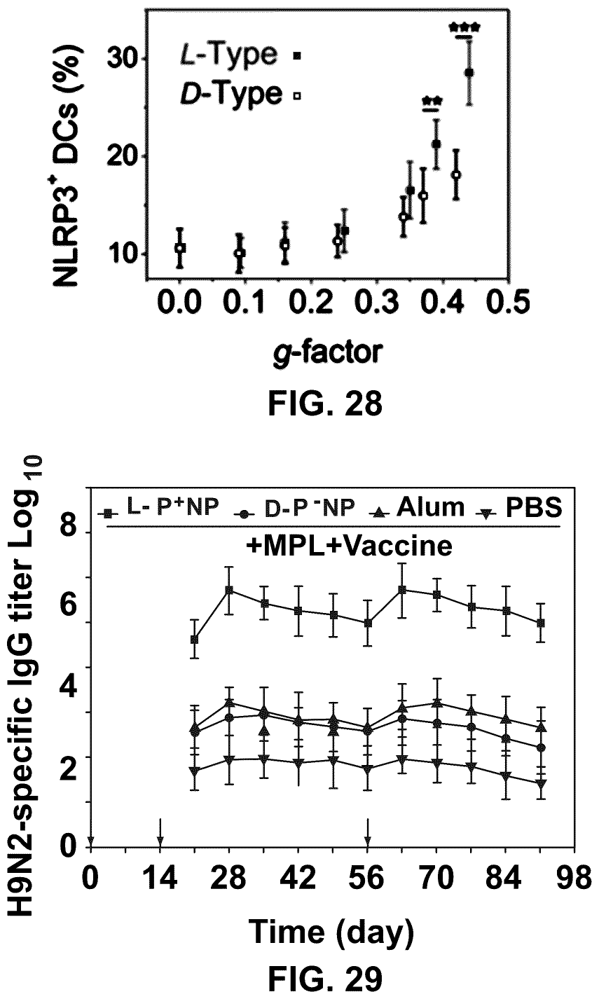

Corresponding in-vivo experiments showed that the L-P⁺NPs and D-P⁻NPs triggered NLRP3 inflammasome activation in the draining lymph nodes of C57BL/6 mice 36 hours after subcutaneous injection of 2 mg of the indicated NPs, MPL (10 kg), and OVA (50 kg). The effect was much greater from L-P⁺NPs and was highly dependent on the chirality of the NP as shown in FIG. 28. At 36 hours after injection the dLNs were collected and the expression of NLRP3 in mouse BMDCs was analyzed by flow cytometry. These data further confirm the data provided herein relating to activation of the inflammasome pathway by NPs, especially by L-P⁺NPs.

Expression of CD40, CD80, CD86, SIINFEKL-MHC I, and MHC II was also markedly elevated after subcutaneous injection of NPs into wild type C57BL/6 mice. By way of contrast, the expression of the same biochemical markers and OVA-specific antibody titers in NLRP3−/− mice were weak after the same NP injections. These data firmly established that the chiral NPs enhanced the immune response through activation of the NLRP3 inflammasome pathway.

Overall, the different stages in immune response to L-P⁺NPs or D-P⁻NPs are described by the schematic in FIG. 20B. First, both types of chiral NPs undergo endocytosis mediated by the receptors CD97 and EMR1. However, the left-handed NP enantiomers associate with these AGPCR proteins much stronger than the right-handed ones. The higher binding affinity of L-P⁺NPs relative to D-P⁻NPs to CD97 and EMR1 is likely due to supramolecular interactions between the chiral extracellular domains made in both cases from EGF-like segments and curved chiral NPs. The NPs may also cause clustering of the AGPCR receptors in the membrane. Second, mechanical stress applied to the cellular membrane by the NPs results in activation of mechanosensitive K⁺ efflux channels to the NLRP3 signaling pathway. Third, the stronger binding of L-P⁺NPs to the receptors than D-P⁻NPs leads to greater inflammasome production. The stronger inflammasome activation triggers the enhanced immune responses to L-P⁺NPs enantiomer relative to the D-P⁻NPs enantiomer.

To investigate the significance of nanoscale chirality in system-level biological responses and the potential of L-P⁺NPs and D-P⁻NPs as adjuvants, C57BL/6 mice were injected with H9N2 influenza vaccine mixed with NPs. Specifically, C57BL/6 mice were immunized with H9N2 influenza vaccine (108 ELD₅₀/0.1 mL, 60 μL) plus the indicated adjuvants: 2 mg L-P⁺NP+10 μg MPL, 2 mg of D-P⁻NP+10 μg MPL, 50 μL Alum (Thermo Fisher Scientific, 77161)+10 μg MPL, or PBS+10 μg MPL, for three times (day 0, day 14, and day 56). The H9N2 specific serum IgG titers were collected over 91 day and measured by ELISA kit according to the protocol (JingMei biotechnology). The results are shown in FIG. 29. Note the Y-axis is a logarithmic scale Log₁₀. In agreement with in-vitro and in-vivo data described above, L-P⁺NPs showed a more significant increase of influenza-specific antibody titer compared to D-P⁻NPs; after injection of L-P⁺NPs, the response was 1258-fold higher than after D-P⁻NPs injection and it lasted for as long as 91 days. At all of the measured time points the D-P⁻NPs were not better than Alum and often a bit lower. Note the Y-axis is a logarithmic scale Log₁₀.

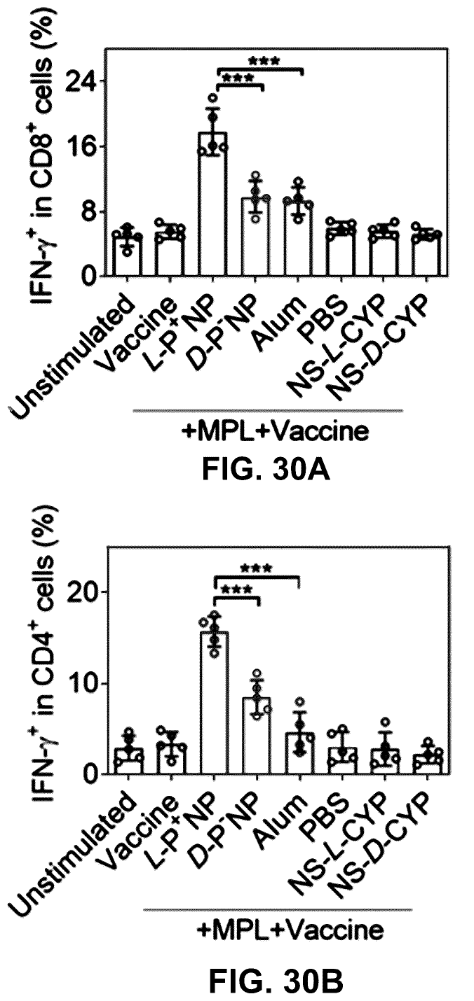

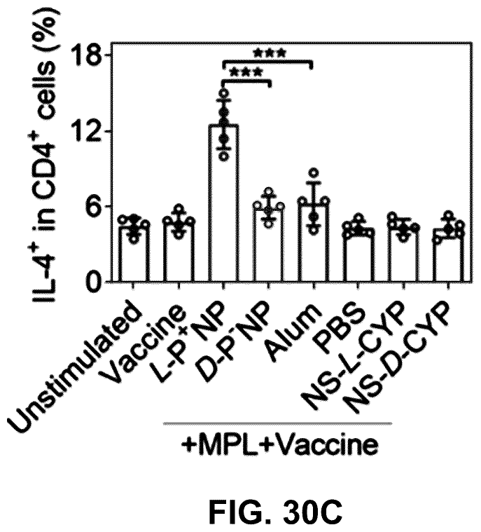

To further investigate the in vivo responses C57BL/6 mice (n=5 per condition) were immunized with H9N2 influenza vaccine with the indicated adjuvants, including MPL, Alum+MPL, D-P⁻NP+MPL, L-P⁺NP+MPL, NS-D-CYP+MPL, or NS-L-CYP+MPL. After 7 days the T cells in spleens were measured by flow cytometry for IFN-γ-secreting CD8+T cells, IFN-γ-secreting CD4⁺ T cells and IL-4-secreting CD4⁺ T cells, data are presented as mean s.d. (n=5). *P<0.05, **P<0.01, ***P<0.001, analyzed by Student's t-test. The proliferation of CD4⁺ T-cells producing IFN-γ (15.68±1.66%), CD8⁺ T-cells producing IFN-γ (17.80±2.88%), and CD4⁺ T-cells producing IL-4 (12.52±1.92%) after activation by L-P⁺NPs in mice spleen were 1.85, 1.81, and 2.11-fold higher, respectively, than by D-P-NPs, see FIGS. 30A, 30B and 30C.

Hyperemia and hyperplasia with inflammatory cell infiltration were observed in D-P⁻NP-treated mice 21 days after being challenged by H9N2 influenza. Severe pulmonary hemorrhage and lung abscess were observed for commercial alum adjuvant or achiral NPs while there was no discernable histopathological lesion found in the mice who received L-P⁺NPs. Therefore, left-handed enantiomers (L-P⁺NPs) bolster the immune response to a greater degree than D-P⁻NPs and can be used to amplify the vaccination effect. The adjuvant performance of chiral NPs was not reduced after coating with DTT, which removes surface dipeptides, thus demonstrating the necessity of nanoscale chirality of the particle as a whole for the enhancement of in-vivo immune response. These findings were verified by negligible immune system activation in multiple control groups as shown in the control groups of FIG. 29 and FIG. 30A-30C.

In-vitro and in-vivo immune responses differ significantly for NP enantiomers and their achiral homologue, which is due to chirality-dependent endocytosis into immune cells. These findings, furthermore, demonstrate the fundamental significance of nanoscale asymmetry and the need for its parametrization in biomedical and toxicological studies regardless of the core NP material. The described chiral effects also open the path to tailoring immune responses using precisely engineered chiral inorganic nanostructures and better understanding of their role in biological systems. Although nanoprism seeds, nanocube seeds and octahedral seeds all were effective in producing chiral NPs according to the present disclosure, it is especially preferred to utilize nanoprism seeds. The nanoprism seeds resulted in the largest absolute g-factors and they are preferred. Although tested only with the influenza vaccine, it is expected that the disclosed chiral nanoparticles will also enhance other vaccines including at least the following vaccines: Covid 19 vaccine; polio vaccine; rubella vaccine; mumps vaccine; chicken pox vaccine; ebola vaccine; hepatitis B vaccine; human papilloma vaccine; the bacille Calmette-Guerin vaccine for tuberculosis; the diphtheria, pertussis (whooping cough), and tetanus (DPT) vaccine; rotavirus vaccine; hepatitis A vaccine; Haemophilus influenzae type b (Hib) disease vaccine; rabies vaccine; the RTS,S/ASO1 vaccine (Mosquirix™); shingrix vaccine; hepatitis C vaccine; HIV vaccines; SIV vaccine; dengue virus vaccine; West Nile virus vaccine; zika virus vaccine; herpes simplex virus vaccine; human cytomegalovirus vaccine; respiratory syncytial virus vaccine; adenovirus vaccine; vesicular stomatitis virus vaccine; encephalomyocarditis virus vaccine; Norovirus vaccine; anthrax vaccine; measles vaccine; typhoid vaccine; cholera vaccine; and diphtheria vaccine. In addition, use of the chiral nanoparticles is also expected to benefit treatment protocols for breast cancer, lung cancer, leukemia, stomach cancer, colorectal cancer, liver cancer, esophagus cancer, mouth cancer, skin cancer, eye cancer, brain cancer, bone cancer, lupus and autoimmune diseases. When used as an adjuvant, the preferred adjuvant effective amount of chiral nanoparticle in a vaccine is from 0.00001 mg to 10 mg per dosage. The preferred weight ratio of antigen to chiral nanoparticle adjuvant in a vaccine is from 1:1000 to 1000:1.

The experimental data in the present disclosure was generated using gold-based chiral nanoparticles; however, it is to be understood that gold as the base is just an example. It is believed that the chiral nanoparticles could be derived from any of the group 10 or 11 elements of the Periodic Table, including gold, silver, nickel, palladium, platinum, and copper. Using the same dipeptide CYP stabilizers and an element appropriate growth solution, chiral nanoparticles could be created from each of these elements and used in the present invention. As discussed herein, the preferred g-factor values for chiral nanoparticles used in the present invention are +/−0.00001 or greater, +/−0.0001 or greater, +/−0.001 or greater, +/−0.01 or greater, more preferably +/−0.1 or greater, more preferably +/−0.2 or greater, more preferably +/−0.3 or greater, most preferably +/−0.4 or greater. The preferred range of values of Hausdorff chirality measure (HCM) for the chiral nanoparticles according to the present invention are greater than 0. The preferred range of values of Osipov-Pickup-Dunmeur chirality index (OPD) for the chiral nanoparticles according to the present invention are for positive values from greater than 0 to less than 20 and for negative values from greater than −20 to less than 0, thus meaning an absolute OPD value of greater than 0 to less than 20. The chirality of the NPs can be determined by any of these three measures.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail. Accordingly, the scope of legal protection afforded this disclosure can only be determined by studying the following claims.

1. A method of producing chiral gold nanoparticles comprising the steps of:
a.) providing a plurality of gold nanoprism seeds having an average edge length of 70 to 80 nm;
b.) preparing a growth solution comprising cetyltrimethylammonium bromide (CTAB), hydrogen tetrchloroaurate (HAuCl₄) and ascorbic acid in distilled water;
c.) adding the nanoprism seeds and a plurality of one dipeptide selected from the group consisting of L-cysteine-phenylalanine and D-cysteine-phenylalanine to the growth solution to form a reaction solution; and
d.) illuminating the reaction solution with right circularly polarized light when selecting as the dipeptide D-cysteine-phenylalanine or left circularly polarized light when selecting as the dipeptide L-cysteine-phenylalanine at a wavelength of 594 nm and an intensity of 84 mW/cm² for a period of time, thereby forming a plurality of chiral nanoparticles having a handedness that is the same as that of the selected dipeptide.

2. The method as recited in claim 1 wherein step d.) comprises using the polarized light for a period of time of 10 to 40 minutes and the produced chiral nanoparticle has a g-factor of +/−0.00001 or greater, or an absolute Osipov-Pickup-Dunmeur chirality index (OPD) value of greater than 0, or a Hausdorff chirality measure (HCM) value of greater than 0.

3. The method as recited in claim 1 wherein step c.) comprises using L-cysteine-phenylalanine as the dipeptide.

4. The method as recited in claim 1 wherein step b.) comprises a growth solution having a molar ratio of cetyltrimethylammonium bromide (CTAB) to hydrogen tetrchloroaurate (HAuCl₄) to ascorbic acid of 4:1:9.5.

5. The method as recited in claim 1 wherein step c.) comprises a ratio of dipeptide to nanoprism seeds of 20 nanomoles of dipeptide to 20 nanomoles of nanoprism seeds.

6. The method as recited in claim 1 further comprising after step d.) the step of centrifuging the reaction solution at 1,600×g for 1 minute, resuspending the pellet in a storage solution, centrifuging the resuspended pellet at 1,600×g for 1 minute and resuspending the pellet in the storage solution, wherein the storage solution is selected from the group consisting of 1 mM CTAB or 5 mM hexadecyl trimethyl ammonium chloride (CTAC) at a level of 50 nM nanoparticles.

7. A vaccine adjuvant comprising a chiral nanoparticle based on a group 10 or group 11 element, said chiral nanoparticle having a g-factor of +/−0.00001 or greater, or an absolute Osipov-Pickup-Dunmeur chirality index (OPD) value of greater than 0, or a Hausdorff chirality measure (HCM) value of greater than 0.

8. The vaccine adjuvant as recited in claim 7 wherein the chiral nanoparticle further comprises surface ligands of poly(ethylene glycol).

9. The vaccine adjuvant as recited in claim 7 wherein the chiral inorganic nanoparticle has size of 1-120 nanometers.

10. A method of inducing an immune response to an antigen in a subject comprising administering to the subject an adjuvant comprising a chiral nanoparticle based on a group 10 or group 11 element, said chiral nanoparticle having a +/−g-factor of 0.00001 or greater, or an absolute Osipov-Pickup-Dunmeur chirality index (OPD) value of greater than 0, or a Hausdorff chirality measure (HCM) value of greater than 0.

11. The method according to claim 10 wherein the chiral nanoparticle further comprises surface ligands poly(ethylene glycol).

12. The method according to claim 10 wherein the chiral inorganic nanoparticle has a size of 1-120 nanometers.

13. The method according to claim 10 wherein the antigen is selected from the group consisting of influenza, Covid 19, polio, rubella, mumps, chicken, ebola, hepatitis B, human papilloma, tuberculosis, diphtheria, pertussis (whooping cough), tetanus, rotavirus, hepatitis A, Haemophilus influenzae type b (Hib), rabies, RTS,S/ASO1 (Mosquirix™), shingrix, hepatitis C, HIV, SIV, dengue virus, West Nile, zika virus, herpes simplex virus, human cytomegalovirus, respiratory syncytial virus, adenovirus, vesicular stomatitis virus, encephalomyocarditis virus, Norovirus, anthrax, measles, typhoid, cholera, and diphtheria.

14. The method according to claim 10 wherein the antigen is selected from the group consisting of breast cancer, lung cancer, leukemia, stomach cancer, colorectal cancer, liver cancer, esophagus cancer, mouth cancer, skin cancer, eye cancer, brain cancer, bone cancer, lupus and autoimmune diseases.

15. A vaccine composition comprising:
(a) an antigen selected from the group consisting of influenza, Covid 19, polio, rubella, mumps, chicken, ebola, hepatitis B, human papilloma, tuberculosis, diphtheria, pertussis (whooping cough), tetanus, rotavirus, hepatitis A, Haemophilus influenzae type b (Hib), rabies, RTS,S/ASO1 (Mosquirix™), shingrix, hepatitis C, HIV, SIV, dengue virus, West Nile, zika virus, herpes simplex virus, human cytomegalovirus, respiratory syncytial virus, adenovirus, vesicular stomatitis virus, encephalomyocarditis virus, Norovirus, anthrax, measles, typhoid, cholera, and diphtheria; and
(b) an adjuvant comprising a chiral nanoparticle based on a group 10 or group 11 element, said chiral nanoparticle having a g-factor of =/−0.00001 or greater, or an absolute Osipov-Pickup-Dunmeur chirality index (OPD) value of greater than 0, or a Hausdorff chirality measure (HCM) value of greater than 0.

16. The vaccine composition as recited in claim 15 wherein the adjuvant is present in an amount of 0.00001 to 10 milligrams per dose of vaccine composition.

17. The vaccine composition as recited in claim 15 wherein the weight ratio of antigen to adjuvant is from 1:1000 to 1000:1.

18. A method of inducing an immune response in a subject comprising administering a vaccine to the subject in need thereof, said vaccine comprising:
(a) an antigen selected from the group consisting of influenza, Covid 19, polio, rubella, mumps, chicken, ebola, hepatitis B, human papilloma, tuberculosis, diphtheria, pertussis (whooping cough), tetanus, rotavirus, hepatitis A, Haemophilus influenzae type b (Hib), rabies, RTS,S/ASO1 (Mosquirix™), shingrix, hepatitis C, HIV, SIV, dengue virus, West Nile, zika virus, herpes simplex virus, human cytomegalovirus, respiratory syncytial virus, adenovirus, vesicular stomatitis virus, encephalomyocarditis virus, Norovirus, anthrax, measles, typhoid, cholera, and diphtheria; and
(b) an adjuvant comprising a chiral nanoparticle based on a group 10 or group 11 element, said chiral nanoparticle having a g-factor of +/−0.00001 or greater, or an absolute Osipov-Pickup-Dunmeur chirality index (OPD) value of greater than 0, or a Hausdorff chirality measure (HCM) value of greater than 0.

19. The method as recited in claim 18 wherein the adjuvant is present in an amount of 0.00001 to 10 milligrams per dose of vaccine composition.

20. The method as recited in claim 18 wherein the weight ratio of antigen to adjuvant in the vaccine is from 1:1000 to 1000:1.
